SEQUENCE LISTING (25 sequences)

SEQ ID NO: 1 - protein, 340 aa, Thermotoga maritima
SLNDKPVGFASVPTADLPEGTVGGLGGEIVFVRTAEELEKYTTAEGKYVIVVDGTIVFEPKREIKVLSDKTIVGINDAKIVGGGLVIKDAQNVIIRNIHFEGFYMEDDPRGKKYDFDYINVENSHHIWIDHCTFVNGNDGAVDIKKYSNYITVSWCKFVDHDKVSLVGSSDKEDPEQAGQAYKVTYHHNYFKNCIQRMPRIRFGMAHVFNNFYSMGLRTGVSGNVFPIYGVASAMGAKVHVEGNYFMGYGAVMAEAGIAFLPTRIMGPVEGYLTLGEGDAKNEFYYCKEPEVRPVEEGKPALDPREYYDYTLDPVQDVPKIVVDGAGAGKLVFEELNTAQ

SEQ ID NO: 2 - DNA, 1149 bp, Artificial Sequence
  Synthetic
atgaaacaacaaaaacggctttacgcccgattgctgacgctgttatttgcgctcatcttcttgctgcctcattctgcagccgcggcatctctcaatgacaaacctgtgggatttgcatccgtaccgacggcggatttaccggagggcacagttggtggattgggtggtgagatcgttttcgtcagaacagcggaagaactggagaaatacacaacggcagaaggaaagtacgtaatagtcgttgatggaacgatcgtttttgaaccaaagagagaaattaaagttctttcagacaaaacgatcgtgggaataaacgatgcaaagatagtcggtggaggtcttgtgataaaggatgcccagaatgtgatcataagaaatattcattttgagggcttttacatggaggacgatcctcggggtaagaagtatgatttcgactatatcaacgtggaaaattctcatcatatctggatcgaccactgtaccttcgtcaacggcaacgatggtgcagtggatattaaaaaatactcaaactacatcactgtttcctggtgtaaatttgtggatcacgacaaggtctccctcgttggttcctccgacaaagaagatccggaacaggcagggcaggcttacaaggtcacgtaccaccataactacttcaagaactgtattcagagaatgcccagaattagatttggaatggcacacgtgttcaataacttctacagcatgggcctgagaacaggtgtctctggaaacgtcttccccatttacggtgttgcttcagcgatgggagcgaaagtccacgttgaaggaaactacttcatgggatacggtgctgtgatggcagaggcgggaattgcgttccttcccaccagaatcatgggtcccgtggaaggttatctgacgctcggtgaaggagatgcaaagaatgaattttactactgtaaagaacctgaagtgcgtcctgttgaggaaggaaaacccgctctcgatccacgcgagtactacgattacacgcttgatccagttcaagatgttccaaaaatcgttgtagatggagcaggagcagggaaactggtgtttgaagaactaaatacggctcagcggccgccccggcattgccagtcggggatattaaaaagagta

SEQ ID NO: 3 - protein, 383 aa, Artificial Sequence
  Synthetic
MKQQKRLYARLLTLLFALIFLLPHSAAAASLNDKPVGFASVPTADLPEGTVGGLGGEIVFVRTAEELEKYTTAEGKYVIVVDGTIVFEPKREIKVLSDKTIVGINDAKIVGGGLVIKDAQNVIIRNIHFEGFYMEDDPRGKKYDFDYINVENSHHIWIDHCTFVNGNDGAVDIKKYSNYITVSWCKFVDHDKVSLVGSSDKEDPEQAGQAYKVTYHHNYFKNCIQRMPRIRFGMAHVFNNFYSMGLRTGVSGNVFPIYGVASAMGAKVHVEGNYFMGYGAVMAEAGIAFLPTRIMGPVEGYLTLGEGDAKNEFYYCKEPEVRPVEEGKPALDPREYYDYTLDPVQDVPKIVVDGAGAGKLVFEELNTAQRPPRHCQSGILKRV

SEQ ID NO: 4 - DNA, 1116 bp, Thermotoga maritima
  CDS (13)..(1113)
cagtagggagggatgctcatgaggttttctcgtgtggtttctttagtactgcttcttgttttcacagctgttctaactggtgctgtaaaagcttctctcaatgacaaacctgtgggatttgcatccgtaccgacggcggatttaccggagggcacagttggtggattgggtggtgagatcgttttcgtcagaacagcggaagaactggagaaatacacaacagcagaaggaaagtacgtaatagtcgttgatggaacgatcgtttttgaaccaaagagagaaattaaagttctttcagacaaaacgatcgtgggaataaacgatgcaaagatagtcggtggaggtcttgtgataaaggatgcccagaatgtgatcataagaaatattcattttgagggcttttacatggaggacgatcctcggggtaagaagtatgatttcgactatatcaacgtggaaaattctcatcatatctggatcgaccactgtaccttcgtcaacggcaacgatggtgcagtggatattaaaaaatactcaaactacatcactgtttcctggtgtaaatttgtggatcacgacaaggtctccctcgttggttcctccgacaaagaagatccggaacaggcagggcaggcttacaaggtcacgtaccaccataactacttcaagaactgtattcagagaatgcccagaattagatttggaatggcacacgtgttcaataacttctacagcatgggcctgagaacaggtgtctctggaaacgtcttccccatttacggtgttgcttcagcgatgggagcgaaagtccacgttgaaggaaactacttcatgggatacggtgctgtgatggcagaggcgggaattgcgttccttcccaccagaatcatgggtcccgtggaaggttatctgacgctcggtgaaggagatgcaaagaatgaattttactactgtaaagaacctgaagtgcgtcctgttgaggaaggaaaacccgctctcgatccacgcgagtactacgattacacgcttgatccagttcaagatgttccaaaaatcgttgtagatggagcaggagcagggaaactggtgtttgaagaactaaatacggctcagtaa

SEQ ID NO: 5 - protein, 367 aa, Thermotoga maritima
MLMRFSRVVSLVLLLVFTAVLTGAVKASLNDKPVGFASVPTADLPEGTVGGLGGEIVFVRTAEELEKYTTAEGKYVIVVDGTIVFEPKREIKVLSDKTIVGINDAKIVGGGLVIKDAQNVIIRNIHFEGFYMEDDPRGKKYDFDYINVENSHHIWIDHCTFVNGNDGAVDIKKYSNYITVSWCKFVDHDKVSLVGSSDKEDPEQAGQAYKVTYHHNYFKNCIQRMPRIRFGMAHVFNNFYSMGLRTGVSGNVFPIYGVASAMGAKVHVEGNYFMGYGAVMAEAGIAFLPTRIMGPVEGYLTLGEGDAKNEFYYCKEPEVRPVEEGKPALDPREYYDYTLDPVQDVPKIVVDGAGAGKLVFEELNTAQ

SEQ ID NO: 6 - DNA, 1113 bp, Artificial Sequence
  Synthetic
atgaaacaacaaaaacggctttacgcccgattgctgacgctgttatttgcgctcatcttcttgctgcctcattctgcagccgcggcatctctcaatgacaaacctgtgggatttgcatccgtaccgacggcggatttaccggagggcacagttggtggattgggtggtgagatcgttttcgtcagaacagcggaagaactggagaaatacacaacggcagaaggaaagtacgtaatagtcgttgatggaacgatcgtttttgaaccaaagagagaaattaaagttctttcagacaaaacgatcgtgggaataaacgatgcaaagatagtcggtggaggtcttgtgataaaggatgcccagaatgtgatcataagaaatattcattttgagggcttttacatggaggacgatcctcggggtaagaagtatgatttcgactatatcaacgtggaaaattctcatcatatctggatcgaccactgtaccttcgtcaacggcaacgatggtgcagtggatattaaaaaatactcaaactacatcactgtttcctggtgtaaatttgtggatcacgacaaggtctccctcgttggttcctccgacaaagaagatccggaacaggcagggcaggcttacaaggtcacgtaccaccataactacttcaagaactgtattcagagaatgcccagaattagatttggaatggcacacgtgttcaataacttctacagcatgggcctgagaacaggtgtctctggaaacgtcttccccatttacggtgttgcttcagcgatgggagcgaaagtccacgttgaaggaaactacttcatgggatacggtgctgtgatggcagaggcgggaattgcgttccttcccaccagaatcatgggtcccgtggaaggttatctgacgctcggtgaaggagatgcaaagaatgaattttactactgtaaagaacctgaagtgcgtcctgttgaggaaggaaaacccgctctcgatccacgcgagtactacgattacacgcttgatccagttcaagatgttccaaaaatcgttgtagatggagcaggagcagggaaactggtgtttgaagaactaaatacggctcagtgataa

SEQ ID NO: 7 - protein, 369 aa, Artificial Sequence
  Synthetic
MKQQKRLYARLLTLLFALIFLLPHSAAAASLNDKPVGFASVPTADLPEGTVGGLGGEIVFVRTAEELEKYTTAEGKYVIVVDGTIVFEPKREIKVLSDKTIVGINDAKIVGGGLVIKDAQNVIIRNIHFEGFYMEDDPRGKKYDFDYINVENSHHIWIDHCTFVNGNDGAVDIKKYSNYITVSWCKFVDHDKVSLVGSSDKEDPEQAGQAYKVTYHHNYFKNCIQRMPRIRFGMAHVFNNFYSMGLRTGVSGNVFPIYGVASAMGAKVHVEGNYFMGYGAVMAEAGIAFLPTRIMGPVEGYLTLGEGDAKNEFYYCKEPEVRPVEEGKPALDPREYYDYTLDPVQDVPKIVVDGAGAGKLVFEELNTAQ

SEQ ID NO: 8 - DNA, 1113 bp, Artificial Sequence
  Synthetic
atgaaacaacaaaaacggctttacgcccgattgctgacgctgttatttgcgctcatcttcttgctgcctcattctgcagccgcggcatctctcaatgacaaacctgtgggatttgcatccgtaccgacggcggatttaccggagggcacagttggtggattgggtggtgagatcgttttcgtcagaacagcggaagaactggagaaatacacaacggcagaaggaaagtacgtaatagtcgttgatggaacgatcgtttttgaaccaaagagagaaattaaagttctttcagacaaaacgatcgtgggaataaacgatgcaaagatagtcggtggaggtcttgtgataaaggatgcccagaatgtgatcataagaaatattcattttgagggcttttacatggaggacgatcctcggggtaagaagtatgatttcgactatatcaacgtggaaaattctcatcatatctggatcgaccacattaccttcgtcaacggcaacgatggtgcagtggatattaaaaaatactcaaactacatcactgtttcctggaacaaatttgtggatcacgacaaggtctccctcgttggttcctccgacaaagaagatccggaacaggcagggcaggcttacaaggtcacgtaccaccataactacttcaagaacctgattcagagaatgcccagaattagatttggaatggcacacgtgttcaataacttctacagcatgggcctgagaacaggtgtctctggaaacgtcttccccatttacggtgttgcttcagcgatgggagcgaaagtccacgttgaaggaaactacttcatgggatacggtgctgtgatggcagaggcgggaattgcgttccttcccaccagaatcatgggtcccgtggaaggttatctgacgctcggtgaaggagatgcaaagaatgaattttactactgtaaagaacctgaagtgcgtcctgttgaggaaggaaaacccgctctcgatccacgcgagtactacgattacacgcttgatccagttcaagatgttccaaaaatcgttgtagatggagcaggagcagggaaactggtgtttgaagaactaaatacggctcagtgataa

SEQ ID NO: 9 - protein, 369 aa, Artificial Sequence
  Synthetic
MKQQKRLYARLLTLLFALIFLLPHSAAAASLNDKPVGFASVPTADLPEGTVGGLGGEIVFVRTAEELEKYTTAEGKYVIVVDGTIVFEPKREIKVLSDKTIVGINDAKIVGGGLVIKDAQNVIIRNIHFEGFYMEDDPRGKKYDFDYINVENSHHIWIDHITFVNGNDGAVDIKKYSNYITVSWNKFVDHDKVSLVGSSDKEDPEQAGQAYKVTYHHNYFKNLIQRMPRIRFGMAHVFNNFYSMGLRTGVSGNVFPIYGVASAMGAKVHVEGNYFMGYGAVMAEAGIAFLPTRIMGPVEGYLTLGEGDAKNEFYYCKEPEVRPVEEGKPALDPREYYDYTLDPVQDVPKIVVDGAGAGKLVFEELNTAQ

SEQ ID NO: 10 - DNA, 42 bp, Artificial Sequence
  Primer
gtcgccggggcggccgctatcaattggtaactgtatctcagc

SEQ ID NO: 11 - DNA, 64 bp, Artificial Sequence
  Primer
gtcgcccgggagctctgatcaggtaccaagcttgtcgacctgcagaatgaggcagcaagaagat

SEQ ID NO: 12 - DNA, 61 bp, Artificial Sequence
  Primer
gtcggcggccgctgatcacgtaccaagcttgtcgacctgcagaatgaggcagcaagaagat

SEQ ID NO: 13 - DNA, 35 bp, Artificial Sequence
  Primer
gtcggagctctatcaattggtaactgtatctcagc

SEQ ID NO: 14 - DNA, 35 bp, Artificial Sequence
  Primer
aacagctgatcacgactgatcttttagcttggcac

SEQ ID NO: 15 - DNA, 37 bp, Artificial Sequence
  Primer
aactgcagccgcggcacatcataatgggacaaatggg

SEQ ID NO: 16 - DNA, 34 bp, Artificial Sequence
  Primer
aaaccgcggcatctctcaatgacaaacctgtggg

SEQ ID NO: 17 - DNA, 38 bp, Artificial Sequence
  Primer
aaagcggccgctgagccgtatttagttcttcaaacacc

SEQ ID NO: 18 - DNA, 29 bp, Artificial Sequence
  Primer
gggaattcttactgagccgtatttagttc

SEQ ID NO: 19 - DNA, 28 bp, Artificial Sequence
  Primer
ccggatccagtagggagggatgctcatg

SEQ ID NO: 20 - DNA, 41 bp, Artificial Sequence
  Primer
cattctgcagccgcggcatctctcaatgacaaacctgtggg

SEQ ID NO: 21 - DNA, 51 bp, Artificial Sequence
  Primer
catcatggatccgcggccgcttatcactgagccgtatttagttcttcaaac

SEQ ID NO: 22 - DNA, 41 bp, Artificial Sequence; identical to SEQ ID NO: 20
  Primer

SEQ ID NO: 23 - DNA, 117 bp, Artificial Sequence
  Primer
ccaacgagggagaccttgtcgtgatccacaaatttgytccaggaaacagtgatgtagtttgagtattttttaatatccactgcaccatcgttgccgttgacgaaggtawygtggtcg

SEQ ID NO: 24 - DNA, 114 bp, Artificial Sequence
  Primer
ggatcacgacaaggtctccctcgttggttcctccgacaaagaagatccggaacaggcagggcaggcttacaaggtcacgtaccaccataactacttcaagaacctgattcagag

SEQ ID NO: 25 - DNA, 51 bp, Artificial Sequence; identical to SEQ ID NO: 21
  Primer

(19) United States
(12) Patent Application Publication
(10) Pub. No.: US 2003/0087415 A1
(43) Pub. Date: May 8, 2003

(54) Extracellular expression of pectate lyase using Bacillus or Escherichia coli

(75) Inventors: Jens Toenne Andersen, Naerum (DK); Lars Kongsbak, Holte (DK); Martin Schulein, Davis, CA (US); Mads Eskelund Bjornvad, Frederiksberg (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 09/834,231

(22) Filed: Apr. 12, 2001

Related U.S. Application Data
(60) Provisional application No. 60/201,345, filed on May 2, 2000.

(30) Foreign Application Priority Data
Apr. 13, 2000 (DK) PA 2000 00621

(51) Int. Cl. C12N 9/88; C07H 21/04; C12P 21/02; C12N 1/21; C12N 15/74

(52) U.S. Cl. 435/232; 435/69.1; 435/252.31; 435/320.1; 536/23.2

(57) ABSTRACT

The present invention relates to transformed bacterial hosts capable of expressing a pectate lyase enzyme endogenous to a strain of Thermotoga maritima, especially a Bacillus or E. coli host cell, is useful in a method for producing the Thermotoga maritima pectate lyase. The Thermotoga maritima pectate lyase is useful for industrial use, e.g. for treatment of textiles.

### CROSS-REFERENCE TO RELATED APPLICATIONS

This application claims, under 35 U.S.C. 119, priority from or the benefit of Danish application no. PA 2000 00621 filed Apr. 13, 2001, and U.S. provisional application No. 60/201,345, filed May 2, 2000 the contents of which are fully incorporated herein by reference.

### BACKGROUND OF THE INVENTION

1. Field of the Invention

The present invention relates to a transformed Bacillus or E. coli host capable of expressing an enzyme having pectate lyase activity which enzyme is endogenous to a strain of Thermotoga maritima, a Bacillus or E. coli expression vector, a method for producing the Thermotoga maritima pectate lyase in a Bacillus or E. coli host cell and a pectate lyase enzyme derived from a strain of Thermotoga maritime or a site-directed variant or mutant of this enzyme.

2. Description of Related Art

Pectin polymers are important constituents of plant cell walls. Pectin is a hetero-polysaccharide with a backbone composed of alternating homogalacturonan (smooth regions) and rhamnogalacturonan (hairy regions). The smooth regions are linear polymers of 1,4-linked alpha-D-galacturonic acid. The galacturonic acid residues can be methyl-esterified on the carboxyl group to a varying degree, usually in a non-random fashion with blocks of polygalacturonic acid being completely methyl-esterified.

Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by the Enzyme Nomenclature (1992) such as pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82).

Pectate lyases have been cloned from different bacterial genera such as Erwinia, Pseudomonas, Klebsiella and Xanthomonas. Cloning of a pectate lyase has also been described from Bacillus subtilis (Nasser et al. (1993) FEBS 335:319-326) and Bacillus sp. YA-14 (Kim et al. (1994) Biosci. Biotech. Biochem. 58:947-949). Purification of pectate lyases with maximum activity in the pH range of 8-10 produced by Bacillus pumilus (Dave and Vaughn (1971) J. Bacteriol. 108:166-174), B. polymyxa (Nagel and Vaughn (1961) Arch. Biochem. Biophys. 93:344-352), B. stearothermophilus (Karbassi and Vaughn (1980) Can. J. Microbiol. 26:377-384), Bacillus sp. (Hasegawa and Nagel (1966) J. Food Sci. 31:838-845) and Bacillus sp. RK9 (Kelly and Fogarty (1978) Can. J. Microbiol. 24:1164-1172) has been reported, however, no publication was found on cloning of pectate lyase encoding genes from these organisms. All the pectate lyases described require divalent cations for maximum activity, calcium ions being the most stimulating.

The international patent application published as WO 99/27083 discloses a pectate lyase cloned from Bacillus licheniformis. The international patent application published as WO 99/27084 discloses pectate lyases cloned from Bacillus agaradhaerens, Bacillus halodurans and other Bacillus sp.

Nelson et al., Nature, 399:323-329 (1999): Evidence for lateral gene transfer between Archaea and Bacteria from genome sequence of Thermotoga maritima” disclose the complete genome sequence of Thermotoga maritima and the derived amino acid sequence. EMBL database entry (AE001722; SPTREMBL:Q9WYR4) suggests the product of the translated amino acid sequence from section 34 of 136 of the complete Thermotoga maritima genome to be a pectate lyase.

It is the object of the present invention to provide a pectate lyase enzyme having high performance in industrial processes, especially high temperature processes, a method for producing such a pectate lyase in high yields, preferably by means of a conventional fermentation technique involving extracellular production of the pectate lyase enzyme endogenous to a strain of Thermotoga maritima which technique makes the use of pectate lyase in industrial applications economically feasible.

### SUMMARY OF THE INVENTION

The inventors have now found that a species of Thermotoga maritima produces an enzyme having pectate lyase (EC 4.2.2.2) activity and have succeeded in cloning and expressing the pectate lyase enzyme in a Bacillus and an Escherichia coli host.

Accordingly, in a first aspect the present invention relates to a Bacillus host transformed with a vector comprising a DNA sequence from Thermotoga maritima encoding for a pectate lyase polypeptide and capable of expressing the DNA sequence.

In a second aspect the present invention relates to a Bacillus expression vector carrying an inserted DNA sequence from Thermotoga maritima encoding for a pectate lyase polypeptide.

In a third aspect, the invention relates to a method for producing, in a Bacillus host cell, a polypeptide having pectate lyase activity, the method comprising the steps of

(a) growing under conditions to overproduce pectate lyase polypeptide in a nutrient medium Bacillus host cells which have been transformed with an expression cassette which includes, as operably joined components,

(i) a transcriptional and translational initiation regulatory region,

(ii) a DNA sequence encoding the pectate lyase polypeptide,

(iii) a transcriptional and translational termination regulatory region, wherein the regulatory regions are functional in the host, and

(iv) a selection marker gene for selecting transformed host cells; and

(b) recovering the pectate lyase polypeptide.

Further, in its fourth aspect, the present invention relates to an enzyme having pectate lyase (EC 4.2.2.2) activity, which enzyme is endogenous to a strain of Thermotoga maritima or a variant of this enzyme wherein one, two, three or four cysteine residues have been altered to other amino acid residues.

### DETAILED DESCRIPTION OF THE INVENTION

Microbial Sources

For the purpose of the present invention the term “obtained from” or “obtainable from” as used herein in connection with a specific source, means that the enzyme is produced or can be produced by the specific source, or by a cell in which a gene from the source have been inserted.

It is at present contemplated that the pectate lyase of the invention may be obtained from a strain of Thermotoga maritima.

In a preferred embodiment, the pectate lyase of the invention is obtained from the species Thermotoga maritima, DSM 3109, this strain being publicly available from Deutsche Sammiung von Mikroorganismen und Zellkulturen GmbH, Mascheroder Weg 1b, D-38124 Braunschweig, Federal Republic of Germany (DSM). It is at present contemplated that a DNA sequence encoding an enzyme with an amino acid sequence identity of at least 85% to the enzyme of the invention may be obtained from other strains belonging to the species Thermotoga maritime.

In the present context, the term “enzyme preparation” is intended to mean either be a conventional enzymatic fermentation product, possibly isolated and purified, from a single species of a microorganism, such preparation usually comprising a number of different enzymatic activities; or a mixture of monocomponent enzymes, preferably enzymes derived from bacterial or fungal species by using conventional recombinant techniques, which enzymes have been fermented and possibly isolated and purified separately and which may originate from different species, preferably fungal or bacterial species; or the fermentation product of a microorganism which acts as a host cell for expression of a recombinant pectate lyase, but which microorganism simultaneously produces other enzymes, e.g. xyloglucanases, proteases, or cellulases, being naturally occurring fermentation products of the microorganism, i.e. the enzyme complex conventionally produced by the corresponding naturally occurring microorganism.

In the present context the term “expression vector” denotes a DNA molecule, linear or circular, that comprises a segment encoding a polypeptide of interest operably linked to additional segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. The expression vector of the invention may be any expression vector that is conveniently subjected to recombinant DNA procedures, and the choice of vector will often depend on the host cell into which the vector is to be introduced. Thus, the vector may be an autonomously replicating vector, i.e. a vector which exists as an extra-chromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The term “recombinant expressed” or “recombinantly expressed” used herein in connection with expression of a polypeptide or protein is defined according to the standard definition in the art. Recombinant expression of a protein is generally performed by using an expression vector as described immediately above.

The term “isolated”, when applied to a polynucleotide molecule, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985). The term “an isolated polynucleotide” may alternatively be termed “a cloned polynucleotide”.

When applied to a protein/polypeptide, the term “isolated” indicates that the protein is found in a condition other than its native environment. In a preferred form, the isolated protein is substantially free of other proteins, particularly other homologous proteins (i.e. “homologous impurities” (see below)). It is preferred to provide the protein in a greater than 40% pure form, more preferably greater than 60% pure form.

Even more preferably it is preferred to provide the protein in a highly purified form, i.e., greater than 80% pure, more preferably greater than 95% pure, and even more preferably greater than 99% pure, as determined by SDS-PAGE.

The term “isolated protein/polypeptide may alternatively be termed “purified protein/polypeptide”.

The term “homologous impurities” means any impurity (e.g. another polypeptide than the polypeptide of the invention) originating from the homologous cell from which the polypeptide of the invention is originally obtained.

The term “obtained from” as used herein in connection with a specific microbial source, means that the polynucleotide and/or polypeptide produced by the specific source, or by a cell in which a gene from the source have been inserted.

The term “operably linked”, when referring to DNA segments, denotes that the segments are arranged so that they function in concert for their intended purposes, e.g. transcription initiates in the promoter and proceeds through the coding segment to the terminator.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

The term “complements of polynucleotide molecules” denotes polynucleotide molecules having a complementary base sequence and reverse orientation as compared to a reference sequence. For example, the sequence 5′ ATGCACGGG 3′ is complementary to 5′ CCCGTGCAT 3′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “promoter” denotes a portion of a gene containing DNA sequences that provide for the binding of RNA polymerase and initiation of transcription. Promoter sequences are commonly, but not always, found in the 5′ non-coding regions of genes.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger peptide is commonly cleaved to remove the secretory peptide during transit through the secretory pathway.

Polynucleotides

Within preferred embodiments of the invention an isolated polynucleotide of the invention will hybridize to similar sized regions of SEQ ID NO:2, 4, 6 or 8, or a sequence complementary thereto, under at least medium stringency conditions.

In particular polynucleotides of the invention will hybridize to a denatured double-stranded DNA probe comprising either the full sequence shown in SEQ ID NO:2, 4, 6 or 8 or the sequence shown in positions 88-1107 of SEQ ID NO:2, 4, 6 or 8 or any probe comprising a subsequence of SEQ ID NO:2, 4, 6 or 8 having a length of at least about 100 base pairs under at least medium stringency conditions, but preferably at high stringency conditions as described in detail below. Suitable experimental conditions for determining hybridization at medium or high stringency between a nucleotide probe and a homologous DNA or RNA sequence involve presoaking of the filter containing the DNA fragments or RNA to hybridize in 5× SSC (Sodium chloride/Sodium citrate, Sambrook et al. 1989) for 10 min, and prehybridization of the filter in a solution of 5× SSC, 5× Denhardt's solution (Sambrook et al. 1989), 0.5% SDS and 100 μg/ml of denatured sonicated salmon sperm DNA (Sambrook et al. 1989), followed by hybridization in the same solution containing a concentration of 10 ng/ml of a random-primed (Feinberg, A. P. and Vogelstein, B. (1983) Anal. Biochem. 132:6-13), 32P-dCTP-labeled (specific activity higher than 1×10⁹ cpm/pg) probe for 12 hours at ca. 45° C. The filter is then washed twice for 30 minutes in 2× SSC, 0.5% SDS at least 60° C. (medium stringency), still more preferably at least 65° C. (medium/high stringency), even more preferably at least 70° C. (high stringency), and even more preferably at least 75° C. (very high stringency).

Molecules to which the oligonucleotide probe hybridizes under these conditions are detected using an X-ray film.

As previously noted, the isolated polynucleotides of the present invention include DNA and RNA. Methods for isolating DNA and RNA are well known in the art. DNA and RNA encoding genes of interest can be cloned in Gene Banks or DNA libraries by means of methods known in the art.

Polynucleotides encoding polypeptides having pectate lyase activity of the invention are then identified and isolated by, for example, hybridization or PCR.

The present invention further provides counterpart polypeptides and polynucleotides from different bacterial strains (orthologs or paralogs). Of particular interest are pectate lyase polypeptides from strains of Thermotoga maritima, examplified by the strain DSM 3109.

Species homologues of a polypeptide with pectate lyase activity of the invention can be cloned using information and compositions provided by the present invention in combination with conventional cloning techniques. For example, a DNA sequence of the present invention can be cloned using chromosomal DNA obtained from a cell type that expresses the protein. Suitable sources of DNA can be identified by probing Northern blots with probes designed from the sequences disclosed herein. A library is then prepared from chromosomal DNA of a positive cell line. A DNA sequence of the invention encoding an polypeptide having pectate lyase activity can then be isolated by a variety of methods, such as by probing with probes designed from the sequences disclosed in the present specification and claims or with one or more sets of degenerate probes based on the disclosed sequences. A DNA sequence of the invention can also be cloned using the polymerase chain reaction, or PCR (Mullis, U.S. Pat. No. 4,683,202), using primers designed from the sequences disclosed herein. Within an additional method, the DNA library can be used to transform or transfect host cells, and expression of the DNA of interest can be detected with an antibody (monoclonal or polyclonal) raised against the pectate lyase cloned from Thermotoga maritima, e.g. from DSM 3109, expressed and purified as described in Materials and Methods and the examples, or by an activity test relating to a polypeptide having pectate lyase activity.

Polypeptides

The sequence of amino acids in positions 30-359 of SEQ ID NO: 3, 5, 7 and 9, respectively, is a mature pectate lyase sequence comprising the catalytic active domain of the enzyme of the invention.

The present invention also provides pectate lyase polypeptides that are substantially homologous to the polypeptide of amino acids in position 30-369 of SEQ ID NO:3 and species homologs (paralogs or orthologs) thereof. The term “substantially homologous” is used herein to denote polypeptides having 85%, preferably at least 88%, more preferably at least 90%, and even more preferably at least 95%, sequence identity to the sequence shown in amino acids nos. 30-369 of SEQ ID NO:3 or its orthologs or paralogs. Such polypeptides will more preferably be at least 98% identical to the sequence shown in amino acids in positions 30-369 of SEQ ID NO:3 or its orthologs or paralogs. Percent sequence identity is determined by conventional methods, by the Clustal method (Thompson, J. D., Higgins, D. G., and Gibson, T. J., (1994), Nucleic Acids Research 22, 4673-4680) with the default settings of the Megalign program in the Lasergene package (DNAstart Inc., 1228 South Park Street, Madison, Wis. 53715). The settings for multiple alignment are: GAP penalty of 10, and GAP length penalty 10; while the pair-wise alignment parameters are GAP penalty of 3 and Ktuple of 1.

Sequence identity of polynucleotide molecules is determined by the Clustal method (Thompson, J. D., Higgins, D. G., and Gibson, T. J., (1994), Nucleic Acids Research 22, 4673-4680) with the default settings of the Megalign program in the Lasergene package (DNAstart Inc., 1228 South Park Street, Madison, Wis. 53715). The settings for multiple alignment are: GAP penalty of 10, and GAP length penalty 10; while the pair-wise alignment parameters are GAP penalty of 5 and Ktuple of 2.

Substantially homologous proteins and polypeptides are characterized as having one or more amino acid substitutions, deletions or additions. These changes are preferably of a minor nature, that is conservative amino acid substitutions (see Table 2) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A (Nilsson et al., EMBO J. 4:1075, 1985; Nilsson et al., Methods Enzymol. 198:3, 1991. See, in general Ford et al., Protein Expression and Purification 2: 95-107, 1991, which is incorporated herein by reference. DNAs encoding affinity tags are available from commercial suppliers (e.g., Pharmacia Biotech, Piscataway, N.J.; New England Biolabs, Beverly, Mass.).

However, even though the changes described above preferably are of a minor nature, such changes may also be of a larger nature such as fusion of larger polypeptides of up to 300 amino acids or more both as amino- or carboxyl-terminal extensions to a polypeptide of the invention having pectate lyase activity.

TABLE 1
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and a-methyl serine) may be substituted for amino acid residues of a polypeptide according to the invention. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, or preferably, are commercially available, and include pipecolic acid, thiazolidine carboxylic acid, dehydroproline, 3- and 4-methylproline, and 3,3-dimethylproline.

Essential amino acids in the pectate lyase polypeptides of the present invention can be identified according to procedures known in the art, such as site-directed mutagenesis or alanine-scanning mutagenesis (Cunningham and Wells, Science 244: 1081-1085, 1989). In the latter technique, single alanine mutations are introduced at every residue in the molecule, and the resultant mutant molecules are tested for biological activity (i.e pectate lyase activity) to identify amino acid residues that are critical to the activity of the molecule. See also, Hilton et al., J. Biol. Chem. 271:4699-4708, 1996. The active site of the enzyme or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-312, 1992; Smith et al., J. Mol. Biol. 224:899-904, 1992; Wlodaver et al., FEBS Left. 309:59-64, 1992. The identities of essential amino acids can also be inferred from analysis of homologies with polypeptides related to a polypeptide according to the invention.

Multiple amino acid substitutions can be made and tested using known methods of mutagenesis, recombination and/or shuffling followed by a relevant screening procedure, such as those disclosed by Reidhaar-Olson and Sauer (Science 241:53-57, 1988), Bowie and Sauer (Proc. Natl. Acad. Sci. USA 86:2152-2156, 1989), WO95/17413, or WO 95/22625. Briefly, these authors disclose methods for simultaneously randomizing two or more positions in a polypeptide, or recombination/shuffling of different mutations (WO95/17413, WO95/22625), followed by selecting for functional a polypeptide, and then sequencing the mutagenized polypeptides to determine the spectrum of allowable substitutions at each position. Other methods that can be used include phage display (e.g., Lowman et al., Biochem. 30:10832-10837, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Mutagenesis/shuffling methods as disclosed above can be combined with high-throughput, automated screening methods to detect activity of cloned, mutagenized polypeptides in host cells. Mutagenized DNA molecules that encode active polypeptides can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can identify and/or prepare a variety of polypeptides that are substantially homologous or identical to residues 30 to 369 of SEQ ID NO: 3 and retain the pectate lyase activity of the wild-type protein.

In a preferred embodiment, the present invention provides a variant enzyme of a pectate lyase endogeneous to Thermotoga maritima, the variant being a site directed variant with 3 removed cysteines, cf. Example 7 and 8. Preferably, the pectate lyase variant of the invention has amino acid substitutions in positions 161, 185 and 223 relative to the amino acid numbering of SEQ ID NO: 3.

The pectate lyase enzyme of the invention may, in addition to the enzyme core comprising the catalytically domain, also comprise a cellulose binding domain (CBD), the cellulose binding domain and enzyme core (the catalytically active domain) of the enzyme being operably linked. The cellulose binding domain (CBD) may exist as an integral part the encoded enzyme, or a CBD from another origin may be introduced into the pectate lyase thus creating an enzyme hybrid. In this context, the term “cellulose-binding domain” is intended to be understood as defined by Peter Tomme et al. “Cellulose-Binding Domains: Classification and Properties” in “Enzymatic Degradation of Insoluble Carbohydrates”, John N. Saddler and Michael H. Penner (Eds.), ACS Symposium Series, No. 618, 1996. This definition classifies more than 120 cellulose-binding domains into 10 families (I-X), and demonstrates that CBDs are found in various enzymes such as cellulases, xylanases, mannanases, arabinofuranosidases, acetyl esterases and chitinases. CBDs have also been found in algae, e.g. the red alga Porphyra purpurea as a non-hydrolytic polysaccharide-binding protein, see Tomme et al., op.cit. However, most of the CBDs are from cellulases and xylanases, CBDs are found at the N and C termini of proteins or are internal. Enzyme hybrids are known in the art, see e.g. WO 90/00609 and WO 95/16782, and may be prepared by transforming into a host cell a DNA construct comprising at least a fragment of DNA encoding the cellulose-binding domain ligated, with or without a linker, to a DNA sequence encoding the pectate lyase and growing the host cell to express the fused gene. Enzyme hybrids may be described by the following formula:

CBD-MR-X

wherein CBD is the N-terminal or the C-terminal region of an amino acid sequence corresponding to at least the cellulose-binding domain; MR is the middle region (the linker), and may be a bond, or a short linking group preferably of from about 2 to about 100 carbon atoms, more preferably of from 2 to 40 carbon atoms; or is preferably from about 2 to about 100 amino acids, more preferably of from 2 to 40 amino acids; and X is an N-terminal or C-terminal region of a polypeptide encoded by the polynucleotide molecule of the invention.

Immunological Cross-Reactivity

Polyclonal antibodies, especially monospecific polyclonal antibodies, to be used in determining immunological cross-reactivity may be prepared by use of a purified enzyme having pectate lyase activity. More specifically, antiserum against the pectate lyase of the invention may be raised by immunizing rabbits (or other rodents) according to the procedure described by N. Axelsen et al. in: A Manual of Quantitative Immunoelectrophoresis, Blackwell Scientific Publications, 1973, Chapter 23, or A. Johnstone and R. Thorpe, Immunochemistry in Practice, Blackwell Scientific Publications, 1982 (more specifically p. 27-31). Purified immunoglobulins may be obtained from the antisera, for example by salt precipitation ((NH₄)₂ SO₄), followed by dialysis and ion exchange chromatography, e.g. on DEAE-Sephadex. Immunochemical characterization of proteins may be done either by Outcherlony double-diffusion analysis (O. Ouchterlony in: Handbook of Experimental Immunology (D. M. Weir, Ed.), Blackwell Scientific Publications, 1967, pp. 655-706), by crossed immunoelectrophoresis (N. Axelsen et al., supra, Chapters 3 and 4), or by rocket immunoelectrophoresis (N. Axelsen et al., Chapter 2).

The Vector

As described in further detail below, the host of the invention is transformed with a vector comprising a pectate lyase encoding DNA sequence. Preferably, the vector is integrated into the genome of the host, more preferably it has been amplified on the genome.

In another preferred embodiment of the invention, the vector is present as an expression plasmid, preferably as a multi-copy plasmid.

The Bacillus expression vector of the invention carries an inserted pectate lyase-encoding DNA sequence. Preferably, the expression cassette of the vector comprises regulatory regions from a Bacillus sp., more preferably are such regulatory regions endogenous to the host.

In another aspect, the present invention further relates to a method for optimisation of pectate lyase enzyme expression in a Bacillus host, the method comprising the steps of expression in the host of a pectate lyase enzyme fused to a reporter molecule; and monitoring the concentration of expressed enzyme in the supernatant of the fermented host by measuring the intrinsic property or properties of the reporter molecule.

In a preferred embodiment, the reporter molecule is a Green Fluorescent Protein, and the intrinsic property is fluorescence emission.

In its fifth and sixth aspect, the invention relates to a polypeptide hybrid consisting essentially of a pectate lyase enzyme fused to a green fluorescent protein, and to a method of producing such a hybrid by expression in a Bacillus host, growth of the transformed host under conditions whereby the transformed culture is substantially free of untransformed cells; incubation of the transformed culture in a nutrient medium, whereby the hybrid is overproduced; and recovery of the hybrid.

Expression of a Pectate Lyase Enzyme

Recombinant Expression Vectors

A recombinant vector comprising a DNA construct encoding the enzyme of the invention may be any vector conveniently subjected to recombinant DNA procedures, and the choice of vector will often depend on the host cell into which it is to be introduced. This introduction of vector into the host cell is often referred to as the transformed host cell. Such transformation indicates introduction of DNA into a host cell by using e.g. protoplasts, natural competent cells, transfection, conjugation, electroporation, or any equivalent method. Thus, the vector may be an autonomously replicating vector, i.e. a vector existing as an extra-chromosomal entity, the replication of which is independent of chromosomal replication, e.g. a plasmid. Alternatively, the vector may be one which, when introduced into a host cell, is integrated into the host cell genome in part or in its entirety and replicated together with the chromosome(s) into which it has been integrated.

The vector is preferably an expression vector in which the DNA sequence encoding the pectate lyase enzyme of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the CBD.

The promoter may be any DNA sequence showing transcriptional activity in the host cell of choice and may be derived from genes encoding proteins either homologous or heterologous to the host cell.

Examples of suitable promoters for use in bacterial host cells include the promoter of the Bacillus stearothermophilus maltogenic amylase gene, the Bacillus licheniformis alpha-amylase gene, the Bacillus amyloliquefaciens alpha-amylase gene, the Bacillus subtilis alkaline protease gen, or the Bacillus pumilus xylosidase gene, or the phage Lambda P_{R} or P_{L} promoters or the E. coli lac, trp or tac promoters. Alternatively, it is possible to design integration vectors such that the DNA encoding the pectate lyase enzyme will only become functionally expressed once it is properly integrated into the host genome, e.g. downstream from a resident promoter.

The DNA sequence encoding the pectate lyase enzyme of the invention may also, if necessary, be operably connected to a suitable terminator.

The recombinant vector of the invention may further comprise a DNA sequence enabling the vector to replicate in the host cell in question.

The vector may also comprise a selectable marker, e.g. a gene the product of which complements a defect in the host cell, or a gene encoding resistance to e.g. antibiotics like kanamycin, chloramphenicol, erythromycin, tetracycline, spectinomycine, or the like, or resistance to heavy metals or herbicides.

To direct a pectate lyase enzyme of the present invention into the secretory pathway of the host cells, a secretory signal sequence (also known as a leader sequence, prepro sequence or pre sequence) may be provided in the recombinant vector. The secretory signal sequence is joined to the DNA sequence encoding the pectate lyase enzyme in the correct reading frame. Secretory signal sequences are commonly positioned 5′ to the DNA sequence encoding the. The secretory signal sequence may be that normally associated with the pectate lyase enzyme or may be from a gene encoding another secreted protein.

The procedures used to ligate the DNA sequences coding for the present pectate tyase enzyme, the promoter and optionally the terminator and/or secretory signal sequence, respectively, or to assemble these sequences by suitable PCR amplification schemes, and to insert them into suitable vectors containing the information necessary for replication or integration, are well known to persons skilled in the art (cf., for instance, Sambrook et al., op.cit.).

Green Fluorescent Protein (GFP) has become a widely used reporter molecule for monitoring gene expression, tracers of cell lineage and as fusion tags for proteins (Crameri et al. (1996); Cubitt et al. (1995); International Patent Application PCT/DK96/00051).

GFP could be fused to pectate lyase enzymes creating a fusion protein having the enzymatic property as well as the fluorescent properties. The expression of this fusion protein could be used to monitor the expressing of pectate lyase enzymes in Bacillus species and hereby be used to optimise expression levels of given pectate lyase enzymes.

Host Cells

The cloned DNA molecule introduced into the host cell may be either homologous or heterologous to the host in question. If homologous to the host cell, i.e. produced by the host cell in nature, it will typically be operably connected to another promoter sequence or, if applicable, another secretory signal sequence and/or terminator sequence than in its natural environment. The term “homologous” is intended to include a DNA sequence encoding an enzyme native to the host organism in question. The term “heterologous” is intended to include a DNA sequence not expressed by the host cell in nature. Thus, the DNA sequence may be from another organism, or it may be a synthetic sequence.

The host cell into which the cloned DNA molecule or the recombinant vector of the invention is introduced may be any cell capable of producing the desired enzyme and includes bacteria, yeast, fungi and higher eukaryotic cells.

Examples of bacterial host cells which on cultivation are capable of producing the enzyme of the invention may be a gram-positive bacteria such as a strain of Bacillus, in particular Bacillus alkalophilus, Bacillus amyloliquefaciens, Bacillus brevis, Bacillus lautus, Bacillus lentus, Bacillus licheniformis, Bacillus circulans, Bacillus coagulans, Bacillus megatherium, Bacillus stearothermophilus, Bacillus subtilis and Bacillus thuringiensis, a strain of Lactobacillus, a strain of Streptococcus, a strain of Streptomyces, in particular Streptomyces lividans and Streptomyces murinus, or the host cell may be a gram-negative bacteria such as a strain of Escherichia coli.

The transformation of the bacteria may be effected by protoplast transformation, electroporation, conjugation, or by using competent cells in a manner known per se (cf. e.g. Sambrook et al., supra).

When expressing the enzyme in a bacteria such as Escherichia coli, the enzyme may be retained in the cytoplasm, typically as insoluble granules (known as inclusion bodies), or may be directed to the periplasmic space by a bacterial secretion sequence. In the former case, the cells are lysed and the granules are recovered and denatured after which the enzyme is refolded by diluting the denaturing agent. In the latter case, the enzyme may be recovered from the periplasmic space by disrupting the cells, e.g. by sonication or osmotic shock, to release the contents of the periplasmic space and recovering the enzyme.

When expressing the enzyme in a gram-positive bacterium such as a strain of Bacillus or a strain of Streptomyces, the enzyme may be retained in the cytoplasm, or may be directed to the extra-cellular medium by a bacterial secretion sequence.

Examples of a fungal host cell which on cultivation are capable of producing the enzyme of the invention is e.g. a strain of Aspergillus or Fusarium, in particular Aspergillus awamori, Aspergillus nidulans, Aspergillus niger, Aspergillus oryzae, and Fusarium oxysporum, and a strain of Trichoderma, preferably Trichoderma harzianum, Trichoderma reesei and Trichoderma viride.

Fungal cells may be transformed by a process involving protoplast formation and transformation of the protoplasts followed by regeneration of the cell wall in a manner known per se. The use of a strain of Aspergillus as a host cell is described in EP 238 023 (Novo Nordisk A/S), the contents of which are hereby incorporated by reference.

Examples of a host cell of yeast origin which on cultivation are capable of producing the enzyme of the invention is e.g. a strain of Hansenula sp., a strain of Kluyveromyces sp., in particular Kluyveromyces lactis and Kluyveromyces marcianus, a strain of Pichia sp., a strain of Saccharomyces, in particular Saccharomyces carlsbergensis, Saccharomyces cerevisae, Saccharomyces kluyveri and Saccharomyces uvarum, a strain of Schizosaccharomyces sp., in particular Schizosaccharomyces pombe, and a strain of Yarrowia sp., in particular Yarrowia lipolytica.

Examples of a host cell of plant origin which on cultivation are capable of producing the enzyme of the invention is e.g. a plant cell of Solanum tuberosum or Nicotiana tabacum.

Method of Producing a Pectate Lyase Enzyme

In another aspect, the present invention also relates to a method of producing the enzyme preparation of the invention, the method comprising culturing a microorganism capable of producing the pectate lyase under conditions permitting the production of the enzyme, and recovering the enzyme from the culture. Culturing may be carried out using conventional fermentation techniques, e.g. culturing in shake flasks or fermentors with agitation to ensure sufficient aeration on a growth medium inducing production of the pectate lyase enzyme. The growth medium may contain a conventional N-source such as peptone, yeast extract or casamino acids, a reduced amount of a conventional C-source such as dextrose or sucrose, and an inducer such as pectinase or composit plant substrates such as cereal brans (e.g. wheat bran or rice husk). The recovery may be carried out using conventional techniques, e.g. separation of bio-mass and supernatant by centrifugation or filtration, recovery of the supernatant or disruption of cells if the enzyme of interest is intracellular, perhaps followed by further purification as described in EP 0 406 314 or by crystallization as described in WO 97/15660.

Further, the present invention provides a method of producing an isolated enzyme according to the invention, wherein a suitable host cell, which has been transformed with a DNA sequence encoding the enzyme, is cultured under conditions permitting the production of the enzyme, and the resulting enzyme is recovered from the culture.

As defined herein, an isolated polypeptide (e.g. an enzyme) is a polypeptide which is essentially free of other polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

The term “isolated polypeptide” may alternatively be termed “purified polypeptide”.

When an expression vector comprising a DNA sequence encoding the enzyme is transformed into a heterologous host cell it is possible to enable heterologous recombinant production of the enzyme of the invention.

Thereby it is possible to make a highly purified or monocomponent pectate lyase composition, characterized in being free from homologous impurities.

In this context, homologous impurities mean any impurities (e.g. other polypeptides than the enzyme of the invention) which originate from the homologous cell where the enzyme of the invention is originally obtained from.

In the present invention the homologous host cell may be a strain of Thermotoga maritima.

The medium used to culture the transformed host cells may be any conventional medium suitable for growing the host cells in question. The expressed pectate lyase enzyme may conveniently be secreted into the culture medium and may be recovered therefrom by well-known procedures including separating the cells from the medium by centrifugation or filtration, precipitating proteinaceous components of the medium by means of a salt such as ammonium sulfate, followed by chromatographic procedures such as ion exchange chromatography, affinity chromatography, or the like.

The present invention also relates to a transgenic plant, plant part or plant cell which has been transformed with a DNA sequence encoding the pectate lyase of the invention so as to express and produce this enzyme in recoverable quantities. The enzyme may be recovered from the plant or plant part.

The transgenic plant can be dicotyledonous or monocotyledonous, for short a dicot or a monocot. Examples of monocot plants are grasses, such as meadow grass (blue grass, Poa), forage grass such as festuca, lolium, temperate grass, such as Agrostis, and cereals, e.g. wheat, oats, rye, barley, rice, sorghum and maize (corn).

Examples of dicot plants are tobacco, legumes, such as lupins, potato, sugar beet, pea, bean and soybean, and cruciferous (family Brassicaceae), such as cauliflower, oil seed rape and the closely related model organism Arabidopsis thaliana.

Examples of plant parts are stem, callus, leaves, root, fruits, seeds, and tubers. In the present context, also specific plant tissues, such as chloroplast, apoplast, mitochondria, vacuole, peroxisomes and cytoplasm are considered to be a plant part. Furthermore, any plant cell, whatever the tissue origin, is considered to be a plant part.

Also included within the scope of the invention are the progeny of such plants, plant parts and plant cells.

The transgenic plant or plant cell expressing the enzyme of the invention may be constructed in accordance with methods known in the art. In short the plant or plant cell is constructed by incorporating one or more expression constructs encoding the enzyme of the invention into the plant host genome and propagating the resulting modified plant or plant cell into a transgenic plant or plant cell.

Conveniently, the expression construct is a DNA construct which comprises a gene encoding the enzyme of the invention in operable association with appropriate regulatory sequences required for expression of the gene in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences is determined, e.g. based on when, where and how the enzyme is desired to be expressed. For instance, the expression of the gene encoding the enzyme of the invention may be constitutive or inducible, or may be developmental, stage or tissue specific, and the gene product may be targeted to a specific tissue or plant part such as seeds or leaves. Regulatory sequences are e.g. described by Tague et al, Plant, Phys., 86, 506, 1988.

For constitutive expression the 35S-CaMV promoter may be used (Franck et al., 1980. Cell 21: 285-294). Organ-specific promoters may eg be a promoter from storage sink tissues such as seeds, potato tubers, and fruits (Edwards & Coruzzi, 1990. Annu. Rev. Genet. 24: 275-303), or from metabolic sink tissues such as meristems (Ito et al., 1994. Plant Mol. Biol. 24: 863-878), a seed specific promoter such as the glutelin, prolamin, globulin or albumin promoter from rice (Wu et al., Plant and Cell Physiology Vol. 39, No. 8 pp. 885-889 (1998)), a Vicia faba promoter from the legumin B4 and the s unknown seed protein gene from Vicia faba described by Conrad U. et al, Journal of Plant Physiology Vol. 152, No. 6 pp. 708-711 (1998), a promotter from a seed oil body protein (Chen et al., Plant and cell physiology vol. 39, No. 9 pp. 935-941 (1998), the storage protein napA promoter from Brassica napus, or any other seed specific promoter known in the art, e.g. as described in WO 91/14772. Furthermore, the promoter may be a leaf specific promoter such as the rbcs promoter from rice or tomato (Kyozuka et al., Plant Physiology Vol. 102, No. 3 pp. 991-1000 (1993), the chlorella virus adenine methyltransferase gene promoter (Mitra, A. and Higgins, D W, Plant Molecular Biology Vol. 26, No. 1 pp. 85-93 (1994), or the aldP gene promoter from rice (Kagaya et al., Molecular and General Genetics Vol. 248, No. 6 pp. 668-674 (1995), or a wound inducible promoter such as the potato pin2 promoter (Xu et al, Plant Molecular Biology Vol. 22, No. 4 pp. 573-588 (1993).

A promoter enhancer element may be used to achieve higher expression of the enzyme in the plant. For instance, the promoter enhancer element may be an intron placed between the promoter and the nucleotide sequence encoding the enzyme. For instance, Xu et al. op cit disclose the use of the first intron of the rice actin 1 gene to enhance expression.

The selectable marker gene and any other parts of the expression construct may be chosen from those available in the art.

The DNA construct is incorporated into the plant genome according to conventional techniques known in the art, including Agrobacterium-mediated transformation, virus-mediated transformation, micro injection, particle bombardment, biolistic transformation, and electroporation (Gasser et al, Science, 244, 1293; Potrykus, Bio/Techn. 8, 535, 1990; Shimamoto et al, Nature, 338, 274, 1989).

Presently, Agrobacterium tumefaciens mediated gene transfer is the method of choice for generating transgenic dicots (for review Hooykas & Schilperoort, 1992. Plant Mol. Biol. 19: 15-38), however it can also be used for transforming monocots, although other transformation methods are generally preferred for these plants. Presently, the method of choice for generating transgenic monocots is particle bombardment (microscopic gold or tungsten particles coated with the transforming DNA) of embryonic calli or developing embryos (Christou, 1992. Plant J. 2: 275-281; Shimamoto, 1994. Curr. Opin. Biotechnol. 5: 158-162; Vasil et al., 1992. Bio/Technology 10: 667-674). An alternative method for transformation of monocots is based on protoplast transformation as described by Omirulleh S, et al., Plant Molecular biology Vol. 21, No. 3 pp. 415-428 (1993).

Following transformation, the transformants having incorporated the expression construct are selected and regenerated into whole plants according to methods well-known in the art.

Enzyme Compositions

In a still further aspect, the present invention relates to an enzyme composition comprising an enzyme exhibiting pectate lyase activity as described above.

The enzyme composition of the invention may, in addition to the pectate lyase of the invention, comprise one or more other enzyme types, for instance hemicellulase such as xylanase and mannanase, cellulase or endo-β-1,4-glucanase components, chitinase, lipase, esterase, pectinase, xyloglucanase, cutinase, phytase, oxidoreductase (peroxidase, haloperoxidase, oxidase, laccase), protease, amylase, reductase, phenoloxidase, ligninase, pullulanase, pectate lyase, pectin acetyl esterase, polygalacturonase, rhamnogalacturonase, pectin lyase, pectin methylesterase, cellobiohydrolase, transglutaminase; or mixtures thereof.

The enzyme composition may be prepared in accordance with methods known in the art and may be in the form of a liquid or a dry composition. For instance, the enzyme composition may be in the form of a granulate or a microgranulate. The enzyme to be included in the composition may be stabilized in accordance with methods known in the art.

Uses

Pectate lyases have potential uses in a lot of different industries and applications. Examples are given below of preferred uses of the enzyme composition of the invention. The dosage of the enzyme composition of the invention and other conditions under which the composition is used may be determined based on methods known in the art.

It is contemplated that the pectate lyase of the invention is useful as an ingredient of a laundry detergent composition or for treatment of textile fabric, especially for scouring of textile or textile fabric at elevated temperatures.

Use in the Detergent Industry

During washing and wearing, dyestuff from dyed fabrics or garment will conventionally bleed from the fabric, which then looks faded and worn. Removal of surface fibers from the fabric will partly restore the original colours and looks of the fabric. By the term “colour clarification”, as used herein, is meant the partly restoration of the initial colours of fabric or garment throughout multiple washing cycles.

The term “de-pilling” denotes removing of pills from the fabric surface.

The term “soaking liquor” denotes aqueous liquor in which laundry may be immersed prior to being subjected to a conventional washing process. The soaking liquor may contain one or more ingredients conventionally used in a washing or laundering process.

The term “washing liquor” denotes aqueous liquor in which laundry is subjected to a washing process, i.e. usually a combined chemical and mechanical action either manually or in a washing machine. Conventionally, the washing liquor is an aqueous solution of a powder or liquid detergent composition.

The term “rinsing liquor” denotes aqueous liquor in which laundry is immersed and treated, conventionally immediately after being subjected to a washing process, in order to rinse the laundry, i.e. essentially remove the detergent solution from the laundry. The rinsing liquor may contain a fabric conditioning or softening composition.

The laundry subjected to the method of the present invention may be conventional washable laundry. Preferably, the major part of the laundry is sewn or unsown fabrics, including knits, wovens, denims, yarns, and towelling, made from cotton, cotton blends or natural or manmade cellulosics (e.g. originating from xylan-containing cellulose fibers such as from wood pulp) or blends thereof. Examples of blends are blends of cotton or rayon/viscose with one or more companion material such as wool, synthetic fibers (e.g. polyamide fibers, acrylic fibers, polyester fibers, polyvinyl alcohol fibers, polyvinyl chloride fibers, polyvinylidene chloride fibers, polyurethane fibers, polyurea fibers, aramid fibers), and cellulose-containing fibers (e.g. rayon/viscose, ramie, flax/linen, jute, cellulose acetate fibers, lyocell).

Detergent Disclosure and Examples

Surfactant System

The detergent compositions according to the present invention comprise a surfactant system, wherein the surfactant can be selected from nonionic and/or anionic and/or cationic and/or ampholytic and/or zwifterionic and/or semi-polar surfactants.

The surfactant is typically present at a level from 0.1% to 60% by weight.

The surfactant is preferably formulated to be compatible with enzyme components present in the composition. In liquid or gel compositions the surfactant is most preferably formulated in such a way that it promotes, or at least does not degrade, the stability of any enzyme in these compositions.

Preferred systems to be used according to the present invention comprise as a surfactant one or more of the nonionic and/or anionic surfactants described herein, preferably sodium alkylether sulphate (AExS).

Polyethylene, polypropylene, and polybutylene oxide conden-sates of alkyl phenols are suitable for use as the nonionic surfactant of the surfactant systems of the present invention, with the polyethylene oxide condensates being pre-ferred. These compounds include the condensation products of alkyl phenols having an alkyl group containing from about 6 to about 14 carbon atoms, preferably from about 8 to about 14 carbon atoms, in either a straight chain or branched-chain configuration with the alkylene oxide. In a preferred embodiment, the ethylene oxide is present in an amount equal to from about 2 to about 25 moles, more preferably from about 3 to about 15 moles, of ethylene oxide per mole of alkyl phenol. Commercially available nonionic surfactants of this type include Igepal™ CO-630, marketed by the GAF Corporation; and Triton X-45, X-114, X-100 and X-102, all marketed by the Rohm & Haas Company. These surfactants are commonly referred to as alkylphenol alkoxylates (e.g., alkyl phenol ethoxylates).

The condensation products of primary and secondary aliphatic alcohols with about 1 to about 25 moles of ethylene oxide are suitable for use as the nonionic surfactant of the nonionic surfactant systems of the present invention. The alkyl chain of the aliphatic alcohol can either be straight or branched, primary or secondary, and generally contains from about 8 to about 22 carbon atoms. Preferred are the condensation products of alcohols having an alkyl group containing from about 8 to about 20 carbon atoms, more preferably from about 10 to about 18 carbon atoms, with from about 2 to about 10 moles of ethylene oxide per mole of alcohol. About 2 to about 7 moles of ethylene oxide and most preferably from 2 to 5 moles of ethylene oxide per mole of alcohol are present in said condensation products. Examples of commercially available nonionic surfactants of this type include Tergitol™ 15-S-9 (The condensation product of C₁₁-C₁₅ linear alcohol with 9 moles ethylene oxide), Tergitol™ 24-L-6 NMW (the condensation product of C₁₂-C₁₄ primary alcohol with 6 moles ethylene oxide with a narrow molecular weight distribution), both marketed by Union Carbide Corporation; Neodol™ 45-9 (the condensation product of C₁₄-C₁₅ linear alcohol with 9 moles of ethylene oxide), Neodol™ 23-3 (the condensation product of C₁₂-C₁₃ linear alcohol with 3.0 moles of ethylene oxide), Neodol™ 45-7 (the condensation product Of C₁₄-C₁₅ linear alcohol with 7 moles of ethylene oxide), Neodol™ 45-5 (the condensation product of C₁₄-C₁₅ linear alcohol with 5 moles of ethylene oxide) marketed by Shell Chemical Company, Kyro™ EOB (the condensation product of C₁₃-C₁₅ alcohol with 9 moles ethylene oxide), marketed by The Procter & Gamble Company, and Genapol LA 050 (the condensation product of C₁₂-C₁₄ alcohol with 5 moles of ethylene oxide) marketed by Hoechst. Preferred range of HLB in these products is from 8-11 and most preferred from 8-10.

Also useful as the nonionic surfactant of the surfactant systems of the present invention are alkylpolysaccharides disclosed in U.S. Pat. No. 4,565,647, having a hydrophobic group containing from about 6 to about 30 carbon atoms, preferably from about 10 to about 16 carbon atoms and a polysaccharide, e.g. a polyglycoside, hydrophilic group containing from about 1.3 to about 10, preferably from about 1.3 to about 3, most preferably from about 1.3 to about 2.7 saccharide units. Any reducing saccharide containing 5 or 6 carbon atoms can be used, e.g., glucose, galactose and galactosyl moieties can be substituted for the glucosyl moieties (optionally the hydrophobic group is attached at the 2-, 3-, 4-, etc. positions thus giving a glucose or galactose as opposed to a glucoside or galactoside). The intersaccharide bonds can be, e.g., between the one position of the additional saccharide units and the 2-, 3-, 4-, and/or 6-positions on the preceding saccharide units.

The preferred alkylpolyglycosides have the formula

R²O(CₙH₂ₙO)ₜ(glycosyl)ₓ

wherein R² is selected from the group consisting of alkyl, alkylphenyl, hydroxyalkyl, hydroxyalkylphenyl, and mixtures thereof in which the alkyl groups contain from about 10 to about 18, preferably from about 12 to about 14, carbon atoms; n is 2 or 3, preferably 2; t is from 0 to about 10, preferably 0; and x is from about 1.3 to about 10, preferably from about 1.3 to about 3, most preferably from about 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4-, and/or 6-position, preferably predominantly the 2-position.

The condensation products of ethylene oxide with a hydrophobic base formed by the condensation of propylene oxide with propylene glycol are also suitable for use as the additional nonionic surfactant systems of the present invention. The hydrophobic portion of these compounds will preferably have a molecular weight from about 1500 to about 1800 and will exhibit water insolubility. The addition of polyoxyethylene moieties to this hydrophobic portion tends to increase the water solubility of the molecule as a whole, and the liquid character of the product is retained up to the point where the polyoxyethylene content is about 50% of the total weight of the condensation product, which corresponds to condensation with up to about 40 moles of ethylene oxide. Examples of compounds of this type include certain of the commercially available Pluronic™ surfactants, marketed by BASF.

Also suitable for use as the nonionic surfactant of the nonionic surfactant system of the present invention, are the condensation products of ethylene oxide with the product resulting from the reaction of propylene oxide and ethylenediamine. The hydrophobic moiety of these products consists of the reaction product of ethylenediamine and excess propylene oxide, and generally has a molecular weight of from about 2500 to about 3000. This hydrophobic moiety is condensed with ethylene oxide to the extent that the condensation product contains from about 40% to about 80% by weight of polyoxyethylene and has a molecular weight of from about 5,000 to about 11,000. Examples of this type of nonionic surfactant include certain of the commercially available Tetronic™ compounds, marketed by BASF.

Preferred for use as the nonionic surfactant of the surfactant systems of the present invention are polyethylene oxide condensates of alkyl phenols, condensation products of primary and secondary aliphatic alcohols with from about 1 to about 25 moles of ethyleneoxide, alkylpolysaccharides, and mixtures hereof. Most preferred are C₈-C₁₄ alkyl phenol ethoxylates having from 3 to 15 ethoxy groups and C₈-C₁₈ alcohol ethoxylates (preferably C₁₀ avg.) having from 2 to 10 ethoxy groups, and mixtures thereof.

Highly preferred nonionic surfactants are polyhydroxy fatty acid amide surfactants of the formula

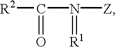

wherein R¹ is H, or R¹ is C₁₋₄ hydrocarbyl, 2-hydroxyethyl, 2-hydroxypropyl or a mixture thereof, R² is C₅₋₃₁ hydrocarbyl, and Z is a polyhydroxyhydrocarbyl having a linear hydrocarbyl chain with at least 3 hydroxyls directly connected to the chain, or an alkoxylated derivative thereof. Preferably, R¹ is methyl, R² is straight C₁₁₋₁₅ alkyl or C₁₆₋₁₈ alkyl or alkenyl chain such as coconut alkyl or mixtures thereof, and Z is derived from a reducing sugar such as glucose, fructose, maltose or lactose, in a reductive amination reaction.

Highly preferred anionic surfactants include alkyl alkoxylated sulfate surfactants. Examples hereof are water soluble salts or acids of the formula RO(A)ₘSO3M wherein R is an unsubstituted C₁₀-C-₂₄ alkyl or hydroxyalkyl group having a C₁₀-C₂₄ alkyl component, preferably a C₁₂-C₂₀ alkyl or hydroxyalkyl, more preferably C₁₂-C₁₈ alkyl or hydroxyalkyl, A is an ethoxy or propoxy unit, m is greater than zero, typically between about 0.5 and about 6, more preferably between about 0.5 and about 3, and M is H or a cation which can be, for example, a metal cation (e.g., sodium, potassium, lithium, calcium, magnesium, etc.), ammonium or substituted-ammonium cation. Alkyl ethoxylated sulfates as well as alkyl propoxylated sulfates are contemplated herein. Specific examples of substituted ammonium cations include methyl-, dimethyl, trimethyl-ammonium cations and quaternary ammonium cations such as tetramethyl-ammonium and dimethyl piperdinium cations and those derived from alkylamines such as ethylamine, diethylamine, triethylamine, mixtures thereof, and the like. Exemplary surfactants are C₁₂-C₁₈ alkyl polyethoxylate (1.0) sulfate (C₁₂-C₁₈E(1.0)M), C₁₂-C₁₈ alkyl polyethoxylate (2.25) sulfate (C₁₂-C₁₈(2.25)M, and C₁₂-C₁₈ alkyl polyethoxylate (3.0) sulfate (C₁₂-C₁₈E(3.0)M), and C₁₂-C₁₈ alkyl polyethoxylate (4.0) sulfate (C₁₂-C₁₈E(4.0)M), wherein M is conveniently selected from sodium and potassium.

Suitable anionic surfactants to be used are alkyl ester sulfonate surfactants including linear esters of C₈-C₂₀ carboxylic acids (i.e., fatty acids) which are sulfonated with gaseous SO₃ according to “The Journal of the American Oil Chemists Society”, 52 (1975), pp. 323-329. Suitable starting materials would include natural fatty substances as derived from tallow, palm oil, etc.

The preferred alkyl ester sulfonate surfactant, especially for laundry applications, comprise alkyl ester sulfonate surfactants of the structural formula:

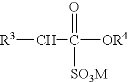

wherein R³ is a C₈-C₂₀ hydrocarbyl, preferably an alkyl, or combination thereof, R⁴ is a C₁-C₆ hydrocarbyl, preferably an alkyl, or combination thereof, and M is a cation which forms a water soluble salt with the alkyl ester sulfonate. Suitable salt-forming cations include metals such as sodium, potassium, and lithium, and substituted or unsubstituted ammonium cations, such as monoethanolamine, diethonolamine, and triethanolamine. Preferably, R³ is C₁₀-C₁₆ alkyl, and R⁴ is methyl, ethyl or isopropyl. Especially preferred are the methyl ester sulfonates wherein R³ is C₁₀-C₁₆ alkyl.

Other suitable anionic surfactants include the alkyl sulfate surfactants which are water soluble salts or acids of the formula ROSO₃M wherein R preferably is a C₁₀-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a cation, e.g., an alkali metal cation (e.g. sodium, potassium, lithium), or ammonium or substituted ammonium (e.g. methyl-, dimethyl-, and trimethyl ammonium cations and quaternary ammonium cations such as tetramethyl-ammonium and dimethyl piperdinium cations and quaternary ammonium cations derived from alkylamines such as ethylamine, diethylamine, triethylamine, and mixtures thereof, and the like). Typically, alkyl chains of C₁₂-C₁₆ are preferred for lower wash temperatures (e.g. below about 50° C.) and C₁₆-C₁₈ alkyl chains are preferred for higher wash temperatures (e.g. above about 50° C.).

Other anionic surfactants useful for detersive purposes can also be included in the laundry detergent compositions of the present invention. Theses can include salts (including, for example, sodium, potassium, ammonium, and substituted ammonium salts such as mono-di- and triethanolamine salts) of soap, C₈-C₂₂ primary or secondary alkanesulfonates, C₈-C₂₄ olefinsulfonates, sulfonated polycarboxylic acids prepared by sulfonation of the pyrolyzed product of alkaline earth metal citrates, e.g., as described in British patent specification No. 1,082,179, C₈-C₂₄ alkylpolyglycolethersulfates (containing up to 10 moles of ethylene oxide); alkyl glycerol sulfonates, fatty acyl glycerol sulfonates, fatty oleyl glycerol sulfates, alkyl phenol ethylene oxide ether sulfates, paraffin sulfonates, alkyl phosphates, isethionates such as the acyl isethionates, N-acyl taurates, alkyl succinamates and sulfosuccinates, monoesters of sulfosuccinates (especially saturated and unsaturated C₁₂-C₁₈ monoesters) and diesters of sulfosuccinates (especially saturated and unsaturated C₆-C₁₂ diesters), acyl sarcosinates, sulfates of alkylpolysaccharides such as the sulfates of alkylpolyglucoside (the nonionic nonsulfated compounds being described below), branched primary alkyl sulfates, and alkyl polyethoxy carboxylates such as those of the formula RO(CH₂CH₂O)ₖ—CH₂COO-M+ wherein R is a C₈-C₂₂ alkyl, k is an integer from 1 to 10, and M is a soluble salt forming cation. Resin acids and hydrogenated resin acids are also suitable, such as rosin, hydrogenated rosin, and resin acids and hydrogenated resin acids present in or derived from tall oil.

Alkylbenzene sulfonates are highly preferred. Especially preferred are linear (straight-chain) alkyl benzene sulfonates (LAS) wherein the alkyl group preferably contains from 10 to 18 carbon atoms.

Further examples are described in “Surface Active Agents and Detergents” (Vol. I and II by Schwartz, Perrry and Berch). A variety of such surfactants are also generally disclosed in U.S. Pat. No. 3,929,678, (Column 23, line 58 through Column 29, line 23, herein incorporated by reference).

When included therein, the laundry detergent compositions of the present invention typically comprise from about 1% to about 40%, preferably from about 3% to about 20% by weight of such anionic surfactants.

The laundry detergent compositions of the present invention may also contain cationic, ampholytic, zwifterionic, and semi-polar surfactants, as well as the nonionic and/or anionic surfactants other than those already described herein.

Cationic detersive surfactants suitable for use in the laundry detergent compositions of the present invention are those having one long-chain hydrocarbyl group. Examples of such cationic surfactants include the ammonium surfactants such as alkyltrimethylammonium halogenides, and those surfactants having the formula:

[R²(OR³)_{y}] [R⁴(OR³)_{y}]₂R⁵N+X—

wherein R² is an alkyl or alkyl benzyl group having from about 8 to about 18 carbon atoms in the alkyl chain, each R³ is selected form the group consisting of —CH₂CH₂—, —CH₂CH(CH₃)—, —CH₂CH(CH₂OH)—, —CH₂CH₂CH₂—, and mixtures thereof; each R⁴ is selected from the group consisting of C₁-C₄ alkyl, C₁-C₄ hydroxyalkyl, benzyl ring structures formed by joining the two R groups, —CH₂CHOHCHOHCOR⁶CHOHCH₂OH, wherein R⁶ is any hexose or hexose polymer having a molecular weight less than about 1000, and hydrogen when y is not 0; R⁵ is the same as R⁴ or is an alkyl chain, wherein the total number of carbon atoms or R²plus R⁵ is not more than about 18; each y is from 0 to about 10, and the sum of the y values is from 0 to about 15; and X is any compatible anion.

Highly preferred cationic surfactants are the water soluble quaternary ammonium compounds useful in the present composition having the formula:

R₁R₂R₃R₄N⁺X⁻C (i)

wherein R₁ is C₈-C₁₆ alkyl, each of R₂, R₃ and R₄ is independently C₁-C₄ alkyl, C₁-C₄ hydroxy alkyl, benzyl, and —(C₂H₄₀)ₓH where x has a value from 2 to 5, and X is an anion. Not more than one of R₂, R₃ or R₄ should be benzyl.

The preferred alkyl chain length for R₁ is C₁₂-C₁₅, particularly where the alkyl group is a mixture of chain lengths derived from coconut or palm kernel fat or is derived synthetically by olefin build up or OXO alcohols synthesis.

Preferred groups for R₂R₃ and R₄ are methyl and hydroxyethyl groups and the anion X may be selected from halide, methosulphate, acetate and phosphate ions.

Examples of suitable quaternary ammonium compounds of formulae (i) for use herein are:

coconut trimethyl ammonium chloride or bromide;

coconut methyl dihydroxyethyl ammonium chloride or bromide;

decyl triethyl ammonium chloride;

decyl dimethyl hydroxyethyl ammonium chloride or bromide;

C₁₂-₁₅ dimethyl hydroxyethyl ammonium chloride or bromide;

coconut dimethyl hydroxyethyl ammonium chloride or bromide;

myristyl trimethyl ammonium methyl sulphate;

lauryl dimethyl benzyl ammonium chloride or bromide;

lauryl dimethyl (ethenoxy)₄ ammonium chloride or bromide;

choline esters (compounds of formula (i) wherein R₁ is

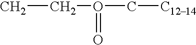

alkyl and R₂R₃R₄ are methyl).

di-alkyl imidazolines [compounds of formula (i)].

Other cationic surfactants useful herein are also described in U.S. Pat. No. 4,228,044 and in EP 000 224.

When included therein, the laundry detergent compositions of the present invention typically comprise from 0.2% to about 25%, preferably from about 1% to about 8% by weight of such cationic surfactants.

Ampholytic surfactants are also suitable for use in the laundry detergent compositions of the present invention. These surfactants can be broadly described as aliphatic derivatives of secondary or tertiary amines, or aliphatic derivatives of heterocyclic secondary and tertiary amines in which the aliphatic radical can be straight- or branched-chain. One of the aliphatic substituents contains at least about 8 carbon atoms, typically from about 8 to about 18 carbon atoms, and at least one contains an anionic water-solubilizing group, e.g. carboxy, sulfonate, sulfate. See U.S. Pat. No. 3,929,678 (column 19, lines 18-35) for examples of ampholytic surfactants.

When included therein, the laundry detergent compositions of the present invention typically comprise from 0.2% to about 15%, preferably from about 1% to about 10% by weight of such ampholytic surfactants.

Zwitterionic surfactants are also suitable for use in laundry detergent compositions. These surfactants can be broadly described as derivatives of secondary and tertiary amines, derivatives of heterocyclic secondary and tertiary amines, or derivatives of quaternary ammonium, quaternary phosphonium or tertiary sulfonium compounds. See U.S. Pat. No. 3,929,678 (column 19, line 38 through column 22, line 48) for examples of zwitterionic surfactants.

When included therein, the laundry detergent compositions of the present invention typically comprise from 0.2% to about 15%, preferably from about 1% to about 10% by weight of such zwitterionic surfactants.

Semi-polar nonionic surfactants are a special category of nonionic surfactants which include water-soluble amine oxides containing one alkyl moiety of from about 10 to about 18 carbon atoms and 2 moieties selected from the group consisting of alkyl groups and hydroxyalkyl groups containing from about 1 to about 3 carbon atoms; watersoluble phosphine oxides containing one alkyl moiety of from about 10 to about 18 carbon atoms and 2 moieties selected from the group consisting of alkyl groups and hydroxyalkyl groups containing from about 1 to about 3 carbon atoms; and water-soluble sulfoxides containing one alkyl moiety from about 10 to about 18 carbon atoms and a moiety selected from the group consisting of alkyl and hydroxyalkyl moieties of from about 1 to about 3 carbon atoms.

Semi-polar nonionic detergent surfactants include the amine oxide surfactants having the formula:

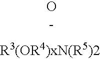

wherein R³ is an alkyl, hydroxyalkyl, or alkyl phenyl group or mixtures thereof containing from about 8 to about 22 carbon atoms; R⁴ is an alkylene or hydroxyalkylene group containing from about 2 to about 3 carbon atoms or mixtures thereof; x is from 0 to about 3: and each R⁵ is an alkyl or hydroxyalkyl group containing from about 1 to about 3 carbon atoms or a polyethylene oxide group containing from about 1 to about 3 ethylene oxide groups. The R⁵ groups can be attached to each other, e.g., through an oxygen or nitrogen atom, to form a ring structure.

These amine oxide surfactants in particular include C₁₀-C₈ alkyl dimethyl amine oxides and C₈-C₁₂ alkoxy ethyl dihydroxy ethyl amine oxides.

When included therein, the laundry detergent compositions of the present invention typically comprise from 0.2% to about 15%, preferably from about 1% to about 10% by weight of such semi-polar nonionic surfactants.

Builder System

The compositions according to the present invention may further comprise a builder system. Any conventional builder system is suitable for use herein including aluminosilicate materials, silicates, polycarboxylates and fatty acids, materials such as ethylenediamine tetraacetate, metal ion sequestrants such as aminopolyphosphonates, particularly ethylenediamine tetramethylene phosphonic acid and diethylene triamine pentamethylenephosphonic acid. Though less preferred for obvious environmental reasons, phosphate builders can also be used herein.

Suitable builders can be an inorganic ion exchange material, commonly an inorganic hydrated aluminosilicate material, more particularly a hydrated synthetic zeolite such as hydrated zeolite A, X, B, HS or MAP.

Another suitable inorganic builder material is layered silicate, e.g. SKS-6 (Hoechst). SKS-6 is a crystalline layered silicate consisting of sodium silicate (Na₂Si₂O₅).

Suitable polycarboxylates containing one carboxy group include lactic acid, glycolic acid and ether derivatives thereof as disclosed in Belgian Patent Nos. 831,368, 821,369 and 821,370. Polycarboxylates containing two carboxy groups include the water-soluble salts of succinic acid, malonic acid, (ethylenedioxy) diacetic acid, maleic acid, diglycollic acid, tartaric acid, tartronic acid and fumaric acid, as well as the ether carboxylates described in German Offenle-enschrift 2,446,686, and 2,446,487, U.S. Pat. No. 3,935,257 and the sulfinyl carboxylates described in Belgian Patent No. 840,623. Polycarboxylates containing three carboxy groups include, in particular, water-soluble citrates, aconitrates and citraconates as well as succinate derivatives such as the carboxymethyloxysuccinates described in British Patent No. 1,379,241, lactoxysuccinates described in Netherlands Application 7205873, and the oxypolycarboxylate materials such as 2-oxa-1,1,3-propane tricarboxylates described in British Patent No. 1,387,447.

Polycarboxylates containing four carboxy groups include oxydisuccinates disclosed in British Patent No. 1,261,829, 1,1,2,2,-ethane tetracarboxylates, 1,1,3,3-propane tetrac7arboxylates containing sulfo substituents include the sulfosuccinate derivatives disclosed in British Patent Nos. 1,398,421 and 1,398,422 and in U.S. Pat. No. 3,936,448, and the sulfonated pyrolysed citrates described in British Patent No. 1,082,179, while polycarboxylates containing phosphone substituents are disclosed in British Patent No. 1,439,000.

Alicyclic and heterocyclic polycarboxylates include cyclopentane-cis,cis-cis-tetracarboxylates, cyclopentadienide pentacarboxylates, 2,3,4,5-tetrahydro-furan-cis, cis, cis-tetracarboxylates, 2,5-tetrahydro-furan-cis, discarboxylates, 2,2,5,5,-tetrahydrofuran-tetracarboxylates, 1,2,3,4,5,6-hexane-hexacarboxylates and carboxymethyl derivatives of polyhydric alcohols such as sorbitol, mannitol and xylitol. Aromatic polycarboxylates include mellitic acid, pyromellitic acid and the phthalic acid derivatives disclosed in British Patent No. 1,425,343.

Of the above, the preferred polycarboxylates are hydroxy-carboxylates containing up to three carboxy groups per molecule, more particularly citrates.

Preferred builder systems for use in the present compositions include a mixture of a water-insoluble aluminosilicate builder such as zeolite A or of a layered silicate (SKS-6), and a water-soluble carboxylate chelating agent such as citric acid.

A suitable chelant for inclusion in the detergent compositions in accordance with the invention is ethylenediamine-N,N′-disuccinic acid (EDDS) or the alkali metal, alkaline earth metal, ammonium, or substituted ammonium salts thereof, or mixtures thereof. Preferred EDDS compounds are the free acid form and the sodium or magnesium salt thereof. Examples of such preferred sodium salts of EDDS include Na₂EDDS and Na₄EDDS. Examples of such preferred magnesium salts of EDDS include MgEDDS and Mg₂EDDS. The magnesium salts are the most preferred for inclusion in compositions in accordance with the invention.

Preferred builder systems include a mixture of a water-insoluble aluminosilicate builder such as zeolite A, and a water soluble carboxylate chelating agent such as citric acid.

Other builder materials that can form part of the builder system for use in granular compositions include inorganic materials such as alkali metal carbonates, bicarbonates, silicates, and organic materials such as the organic phosphonates, amino polyalkylene phosphonates and amino polycarboxylates.

Other suitable water-soluble organic salts are the homo- or co-polymeric acids or their salts, in which the polycarboxylic acid comprises at least two carboxyl radicals separated form each other by not more than two carbon atoms.

Polymers of this type are disclosed in GB-A-1,596,756. Examples of such salts are polyacrylates of MW 2000-5000 and their copolymers with maleic anhydride, such copolymers having a molecular weight of from 20,000 to 70,000, especially about 40,000.

Detergency builder salts are normally included in amounts of from 5% to 80% by weight of the composition. Preferred levels of builder for liquid detergents are from 5% to 30%.

Enzymes

Preferred detergent compositions, in addition to the enzyme preparation of the invention, comprise other enzyme(s) which provides cleaning performance and/or fabric care benefits.

Such enzymes include proteases, lipases, cutinases, amylases, cellulases, peroxidases, oxidases (e.g. laccases).

Proteases: Any protease suitable for use in alkaline solutions can be used. Suitable proteases include those of animal, vegetable or microbial origin. Microbial origin is preferred. Chemically or genetically modified mutants are included. The protease may be a serine protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270.

Preferred commercially available protease enzymes include those sold under the trade names Alcalase, Savinase, Primase, Durazym, and Esperase by Novo Nordisk A/S (Denmark), those sold under the tradename Maxatase, Maxacal, Maxapem, Properase, Purafect and Purafect OXP by Genencor International, and those sold under the tradename Opticlean and Optimase by Solvay Enzymes. Protease enzymes may be incorporated into the compositions in accordance with the invention at a level of from 0.00001% to 2% of enzyme protein by weight of the composition, preferably at a level of from 0.0001% to 1% of enzyme protein by weight of the composition, more preferably at a level of from 0.001% to 0.5% of enzyme protein by weight of the composition, even more preferably at a level of from 0.01% to 0.2% of enzyme protein by weight of the composition.

Lipases: Any lipase suitable for use in alkaline solutions can be used. Suitable lipases include those of bacterial or fungal origin. Chemically or genetically modified mutants are included.

Examples of useful lipases include a Humicola lanuginosa lipase, e.g., as described in EP 258 068 and EP 305 216, a Rhizomucor miehei lipase, e.g., as described in EP 238 023, a Candida lipase, such as a C. antarctica lipase, e.g., the C. antarctica lipase A or B described in EP 214 761, a Pseudomonas lipase such as a P. alcaligenes and P. pseudoalcaligenes lipase, e.g., as described in EP 218 272, a P. cepacia lipase, e.g., as described in EP 331 376, a P. stutzeri lipase, e.g., as disclosed in GB 1,372,034, a P. fluorescens lipase, a Bacillus lipase, e.g., a B. subtilis lipase (Dartois et al., (1993), Biochemica et Biophysica acta 1131, 253-260), a B. stearothermophilus lipase (JP 64/744992) and a B. pumilus lipase (WO 91/16422).

Furthermore, a number of cloned lipases may be useful, including the Penicillium camembertii lipase described by Yamaguchi et al., (1991), Gene 103, 61-67), the Geotricum candidum lipase (Schimada, Y. et al., (1989), J. Biochem., 106, 383-388), and various Rhizopus lipases such as a R. delemar lipase (Hass, M. J et al., (1991), Gene 109,117-113), a R. niveus lipase (Kugimiya et al., (1992), Biosci. Biotech. Biochem. 56, 716-719) and a R. oryzae lipase.

Other types of lipolytic enzymes such as cutinases may also be useful, e.g., a cutinase derived from Pseudomonas mendocina as described in WO 88/09367, or a cutinase derived from Fusarium solani pisi (e.g. described in WO 90/09446).

Especially suitable lipases are lipases such as M1 Lipase™, Luma fast™ and Lipomax™ (Genencor), Lipolase™ and Lipolase Ultra™ (Novo Nordisk A/S), and Lipase P “Amano” (Amano Pharmaceutical Co. Ltd.).

The lipases are normally incorporated in the detergent composition at a level of from 0.00001% to 2% of enzyme protein by weight of the composition, preferably at a level of from 0.0001% to 1% of enzyme protein by weight of the composition, more preferably at a level of from 0.001% to 0.5% of enzyme protein by weight of the composition, even more preferably at a level of from 0.01% to 0.2% of enzyme protein by weight of the composition.

Amylases: Any amylase (a and/or b) suitable for use in alkaline solutions can be used. Suitable amylases include those of bacterial or fungal origin. Chemically or genetically modified mutants are included. Amylases include, for example, a-amylases obtained from a special strain of B. licheniformis, described in more detail in GB 1,296,839. Commercially available amylases are Duramyl™, Termamyl™, Fungamyl™ and BAN™ (available from Novo Nordisk A/S) and Rapidase™ and Maxamyl P™ (available from Genencor).

The amylases are normally incorporated in the detergent composition at a level of from 0.00001% to 2% of enzyme protein by weight of the composition, preferably at a level of from 0.0001% to 1% of enzyme protein by weight of the composition, more preferably at a level of from 0.001% to 0.5% of enzyme protein by weight of the composition, even more preferably at a level of from 0.01% to 0.2% of enzyme protein by weight of the composition.

Cellulases: Any cellulase suitable for use in alkaline solutions can be used. Suitable cellulases include those of bacterial or fungal origin. Chemically or genetically modified mutants are included. Suitable cellulases are disclosed in U.S. Pat. No. 4,435,307 which discloses fungal cellulases produced from Humicola insolens, in WO 96/34108 and WO 96/34092 which disclose bacterial alkalophilic cellulases (BCE 103) from Bacillus, and in WO 94/21801, U.S. Pat. No. 5,475,101 and U.S. Pat. No. 5,419,778 which disclose EG III cellulases from is Trichoderma. Especially suitable cellulases are the cellulases having colour care benefits. Examples of such cellulases are cellulases described in European patent application No. 0 495 257. Commercially available cellulases include Celluzyme™ and Carezyme™ produced by a strain of Humicola insolens (Novo Nordisk A/S), KAC-500(B)™ (Kao Corporation), and Puradax™ (Genencor International).

Cellulases are normally incorporated in the detergent composition at a level of from 0.00001% to 2% of enzyme protein by weight of the composition, preferably at a level of from 0.0001% to 1% of enzyme protein by weight of the composition, more preferably at a level of from 0.001% to 0.5% of enzyme protein by weight of the composition, even more preferably at a level of from 0.01% to 0.2% of enzyme protein by weight of the composition.

Peroxidases/Oxidases: Peroxidase enzymes are used in combination with hydrogen peroxide or a source thereof (e.g. a percarbonate, perborate or persulfate). Oxidase enzymes are used in combination with oxygen. Both types of enzymes are used for “solution bleaching”, i.e. to prevent transfer of a textile dye from a dyed fabric to another fabric when said fabrics are washed together in a wash liquor, preferably together with an enhancing agent as described in e.g. WO 94112621 and WO 95/01426. Suitable peroxidases/oxidases include those of plant, bacterial or fungal origin. Chemically or genetically modified mutants are included.

Peroxidase and/or oxidase enzymes are normally incorporated in the detergent composition at a level of from 0.00001% to 2% of enzyme protein by weight of the composition, preferably at a level of from 0.0001% to 1% of enzyme protein by weight of the composition, more preferably at a level of from 0.001% to 0.5% of enzyme protein by weight of the composition, even more preferably at a level of from 0.01% to 0.2% of enzyme protein by weight of the composition.

Mixtures of the above mentioned enzymes are encompassed herein, in particular a mixture of a protease, an amylase, a lipase and/or a cellulase.

The enzyme of the invention, or any other enzyme incorporated in the detergent composition, is normally incorporated in the detergent composition at a level from 0.00001% to 2% of enzyme protein by weight of the composition, preferably at a level from 0.0001% to 1% of enzyme protein by weight of the composition, more preferably at a level from 0.001% to 0.5% of enzyme protein by weight of the composition, even more preferably at a level from 0.01% to 0.2% of enzyme protein by weight of the composition.

Bleaching Agents

Additional optional detergent ingredients that can be included in the detergent compositions of the present invention include bleaching agents such as PB1, PB4 and percarbonate with a particle size of 400-800 microns. These bleaching agent components can include one or more oxygen bleaching agents and, depending upon the bleaching agent chosen, one or more bleach activators. When present oxygen bleaching compounds will typically be present at levels of from about 1% to about 25%. In general, bleaching compounds are optional added components in non-liquid formulations, e.g. granular detergents.

The bleaching agent component for use herein can be any of the bleaching agents useful for detergent compositions including oxygen bleaches as well as others known in the art.

The bleaching agent suitable for the present invention can be an activated or non-activated bleaching agent.

One category of oxygen bleaching agent that can be used encompasses percarboxylic acid bleaching agents and salts thereof. Suitable examples of this class of agents include magnesium monoperoxyphthalate hexahydrate, the magnesium salt of meta-chloro perbenzoic acid, 4-nonylamino-4-oxoperoxybutyric acid and diperoxydodecanedioic acid. Such bleaching agents are disclosed in U.S. Pat. No. 4,483,781, U.S. Pat. No. 740,446, EP 0 133 354 and U.S. Pat. No. 4,412,934. Highly preferred bleaching agents also include 6-nonylamino-6-oxoperoxycaproic acid as described in U.S. Pat. No. 4,634,551.

Another category of bleaching agents that can be used encompasses the halogen bleaching agents. Examples of hypohalite bleaching agents, for example, include trichloro isocyanuric acid and the sodium and potassium dichloroisocyanurates and N-chloro and N-bromo alkane sulphonamides. Such materials are normally added at 0.5-10% by weight of the finished product, preferably 1-5% by weight.

The hydrogen peroxide releasing agents can be used in combination with bleach activators such as tetra-acetylethylenediamine (TAED), nonanoyloxybenzenesulfonate (NOBS, described in U.S. Pat. No. 4,412,934), 3,5-trimethyl-hexsanoloxybenzenesulfonate (ISONOBS, described in EP 120 591) or pentaacetylglucose (PAG), which are perhydrolyzed to form a peracid as the active bleaching species, leading to improved bleaching effect. In addition, very suitable are the bleach activators C8(6-octanamido-caproyl) oxybenzene-sulfonate, C9(6-nonanamido caproyl) oxybenzenesulfonate and C10 (6-decanamido caproyl) oxybenzenesulfonate or mixtures thereof. Also suitable activators are acylated citrate esters such as disclosed in European Patent Application No. 91870207.7.

Useful bleaching agents, including peroxyacids and bleaching systems comprising bleach activators and peroxygen bleaching compounds for use in cleaning compositions according to the invention are described in application U.S. Ser. No. 08/136,626.

The hydrogen peroxide may also be present by adding an enzymatic system (i.e. an enzyme and a substrate therefore) which is capable of generation of hydrogen peroxide at the beginning or during the washing and/or rinsing process. Such enzymatic systems are disclosed in European Patent Application EP 0 537 381.

Bleaching agents other than oxygen bleaching agents are also known in the art and can be utilized herein. One type of non-oxygen bleaching agent of particular interest includes photoactivated bleaching agents such as the sulfonated zinc and/or aluminium phthalocyanines. These materials can be deposited upon the substrate during the washing process. Upon irradiation with light, in the presence of oxygen, such as by hanging clothes out to dry in the daylight, the sulfonated zinc phthalocyanine is activated and, consequently, the substrate is bleached. Preferred zinc phthalocyanine and a photoactivated bleaching process are described in U.S. Pat. No. 4,033,718. Typically, detergent composition will contain about 0.025% to about 1.25%, by weight, of sulfonated zinc phthalocyanine.

Bleaching agents may also comprise a manganese catalyst. The manganese catalyst may, e.g., be one of the compounds described in “Efficient manganese catalysts for low-temperature bleaching”, Nature 369, 1994, pp. 637-639.

Suds Suppressors

Another optional ingredient is a suds suppressor, exemplified by silicones, and silica-silicone mixtures. Silicones can generally be represented by alkylated polysiloxane materials, while silica is normally used in finely divided forms exemplified by silica aerogels and xerogels and hydrophobic silicas of various types. Theses materials can be incorporated as particulates, in which the suds suppressor is advantageously releasably incorporated in a water-soluble or water-dispersible, substantially non surface-active detergent impermeable carrier. Alternatively the suds suppressor can be dissolved or dispersed in a liquid carrier and applied by spraying on to one or more of the other components.

A preferred silicone suds controlling agent is disclosed in U.S. Pat. No. 3,933,672. Other particularly useful suds suppressors are the self-emulsifying silicone suds suppressors, described in German Patent Application DTOS 2,646,126. An example of such a compound is DC-544, commercially available form Dow Corning, which is a siloxane-glycol copolymer. Especially preferred suds controlling agent are the suds suppressor system comprising a mixture of silicone oils and 2-alkyl-alkanols. Suitable 2-alkyl-alkanols are 2-butyl-octanol which are commercially available under the trade name Isofol 12 R.

Such suds suppressor system are described in European Patent Application EP 0 593 841.

Especially preferred silicone suds controlling agents are described in European Patent Application No. 92201649.8. Said compositions can comprise a silicone/silica mixture in combination with fumed nonporous silica such as Aerosil^{R}.

The suds suppressors described above are normally employed at levels of from 0.001% to 2% by weight of the composition, preferably from 0.01% to 1% by weight.

Other Components

Other components used in detergent compositions may be employed such as soil-suspending agents, soil-releasing agents, optical brighteners, abrasives, bactericides, tarnish inhibitors, coloring agents, and/or encapsulated or nonencapsulated perfumes.

Especially suitable encapsulating materials are water soluble capsules which consist of a matrix of polysaccharide and polyhydroxy compounds such as described in GB 1,464,616.

Other suitable water soluble encapsulating materials comprise dextrins derived from ungelatinized starch acid esters of substituted dicarboxylic acids such as described in U.S. Pat. No. 3,455,838. These acid-ester dextrins are, preferably, prepared from such starches as waxy maize, waxy sorghum, sago, tapioca and potato. Suitable examples of said encapsulation materials include N-Lok manufactured by National Starch. The N-Lok encapsulating material consists of a modified maize starch and glucose. The starch is modified by adding monofunctional substituted groups such as octenyl succinic acid anhydride.

Antiredeposition and soil suspension agents suitable herein include cellulose derivatives such as methylcellulose, carboxymethylcellulose and hydroxyethylcellulose, and homo- or co-polymeric polycarboxylic acids or their salts. Polymers of this type include the polyacrylates and maleic anhydride-acrylic acid copolymers previously mentioned as builders, as well as copolymers of maleic anhydride with ethylene, methylvinyl ether or methacrylic acid, the maleic anhydride constituting at least 20 mole percent of the copolymer. These materials are normally used at levels of from 0.5% to 10% by weight, more preferably form 0.75% to 8%, most preferably from 1% to 6% by weight of the composition.

Preferred optical brighteners are anionic in character, examples of which are disodium 4,4′-bis-(2-diethanolamino-4-anilino-s-triazin-6-ylamino)stilbene-2:2′ disulphonate, disodium 4, -4′-bis-(2-morpholino-4-anilino-s-triazin-6-ylamino-stilbene-2:2′-disulphonate, disodium 4,4′-bis-(2,4-dianilino-s-triazin-6-ylamino)stilbene-2:2′-disulphonate, monosodium 4′,4″-bis-(2,4-dianilino-s-tri-azin-6 ylamino)stilbene-2-sulphonate, disodium 4,4′-bis-(2-anilino-4-(N-methyl-N-2-hydroxyethylamino)-s-triazin-6-ylamino)stilbene-2,2′-disulphonate, di-sodium 4,4′-bis-(4-phenyl-2,1,3-triazol-2-yl)-stilbene-2,2′ disulphonate, di-so-dium 4,4′-bis(2-anilino-4-(1-methyl-2-hydroxyethylamino)-s-triazin-6-ylami-no)stilbene-2,2′disulphonate, sodium 2(stilbyl-4″-(naphtho-1′,2′:4,5)-1,2,3, -triazole-2″-sulphonate and 4,4′-bis(2-sulphostyryl)biphenyl.

Other useful polymeric materials are the polyethylene glycols, particularly those of molecular weight 1000-10000, more particularly 2000 to 8000 and most preferably about 4000. These are used at levels of from 0.20% to 5% more preferably from 0.25% to 2.5% by weight. These polymers and the previously mentioned homo- or co-polymeric poly-carboxylate salts are valuable for improving whiteness maintenance, fabric ash deposition, and cleaning performance on clay, proteinaceous and oxidizable soils in the presence of transition metal impurities.

Soil release agents useful in compositions of the present invention are conventionally copolymers or terpolymers of terephthalic acid with ethylene glycol and/or propylene glycol units in various arrangements. Examples of such polymers are disclosed in U.S. Pat. No. 4,116,885 and 4,711,730 and EP 0 272 033. A particular preferred polymer in accordance with EP 0 272 033 has the formula:

(CH₃(PEG)₄₃)_{0.75}(POH)_{0.25}[T-PO)_{2.8}(T-PEG)_{0.4}]T(POH)_{0.25}((PEG)₄₃CH₃)_{0.75}

where PEG is —(OC₂H₄)O—, PO is (OC₃H₆O) and T is (pOOC₆H₄CO).

Also very useful are modified polyesters as random copolymers of dimethyl terephthalate, dimethyl sulfoisophthalate, ethylene glycol and 1,2-propanediol, the end groups consisting primarily of sulphobenzoate and secondarily of mono esters of ethylene glycol and/or 1,2-propanediol. The target is to obtain a polymer capped at both end by sulphobenzoate groups, “primarily”, in the present context most of said copolymers herein will be endcapped by sulphobenzoate groups. However, some copolymers will be less than fully capped, and therefore their end groups may consist of monoester of ethylene glycol and/or 1,2-propanediol, thereof consist “secondarily” of such species.

The selected polyesters herein contain about 46% by weight of dimethyl terephthalic acid, about 16% by weight of 1,2-propanediol, about 10% by weight ethylene glycol, about 13% by weight of dimethyl sulfobenzoic acid and about 15% by weight of sulfoisophthalic acid, and have a molecular weight of about 3.000. The polyesters and their method of preparation are described in detail in EP 311 342.

Softening Agents

Fabric softening agents can also be incorporated into laundry detergent compositions in accordance with the present invention. These agents may be inorganic or organic in type. Inorganic softening agents are exemplified by the smectite clays disclosed in GB-A-1400898 and in U.S. Pat. No. 5,019,292. Organic fabric softening agents include the water insoluble tertiary amines as disclosed in GB-Al 514 276 and EP 0 011 340 and their combination with mono C₁₂-C₁₄ quaternary ammonium salts are disclosed in EP-B-0 026 528 and di-long-chain amides as disclosed in EP 0 242 919. Other useful organic ingredients of fabric softening systems include high molecular weight polyethylene oxide materials as disclosed in EP 0 299 575 and 0 313 146.

Levels of smectite clay are normally in the range from 5% to 15%, more preferably from 8% to 12% by weight, with the material being added as a dry mixed component to the remainder of the formulation. Organic fabric softening agents such as the water-insoluble tertiary amines or dilong chain amide materials are incorporated at levels of from 0.5% to 5% by weight, normally from 1% to 3% by weight whilst the high molecular weight polyethylene oxide materials and the water soluble cationic materials are added at levels of from 0.1% to 2%, normally from 0.15% to 1.5% by weight. These materials are normally added to the spray dried portion of the composition, although in some instances it may be more convenient to add them as a dry mixed particulate, or spray them as molten liquid on to other solid components of the composition.

Polymeric Dye-Transfer Inhibiting Agents

The detergent compositions according to the present invention may also comprise from 0.001% to 10%, preferably from 0.01% to 2%, more preferably form 0.05% to 1% by weight of polymeric dye-transfer inhibiting agents. Said polymeric dye-transfer inhibiting agents are normally incorporated into detergent compositions in order to inhibit the transfer of dyes from colored fabrics onto fabrics washed therewith. These polymers have the ability of complexing or adsorbing the fugitive dyes washed out of dyed fabrics before the dyes have the opportunity to become attached to other articles in the wash.

Especially suitable polymeric dye-transfer inhibiting agents are polyamine N-oxide polymers, copolymers of N-vinyl-pyrrolidone and N-vinylimidazole, polyvinylpyrrolidone polymers, polyvinyloxazolidones and polyvinylimidazoles or mixtures thereof.

Addition of such polymers also enhances the performance of the enzymes according the invention.

The detergent composition according to the invention can be in liquid, paste, gels, bars or granular forms.

Non-dusting granulates may be produced, e.g., as disclosed in U.S. Pat. No. 4,106,991 and 4,661,452 (both to Novo Industri A/S) and may optionally be coated by methods known in the art. Examples of waxy coating materials are poly(ethylene oxide) products (polyethyleneglycol, PEG) with mean molecular weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

Granular compositions according to the present invention can also be in “compact form”, i.e. they may have a relatively higher density than conventional granular detergents, i.e. form 550 to 950 g/l; in such case, the granular detergent compositions according to the present invention will contain a lower amount of “Inorganic filler salt”, compared to conventional granular detergents; typical filler salts are alkaline earth metal salts of sulphates and chlorides, typically sodium sulphate; “Compact” detergent typically comprise not more than 10% filler salt. The liquid compositions according to the present invention can also be in “concentrated form”, in such case, the liquid detergent compositions according to the present invention will contain a lower amount of water, compared to conventional liquid detergents. Typically, the water content of the concentrated liquid detergent is less than 30%, more preferably less than 20%, most preferably less than 10% by weight of the detergent compositions.

The compositions of the invention may for example, be formulated as hand and machine laundry detergent compositions including laundry additive compositions and compositions suitable for use in the pretreatment of stained fabrics, rinse added fabric softener compositions, and compositions for use in general household hard surface cleaning operations and dishwashing operations.

The following examples are meant to exemplify compositions for the present invention, but are not necessarily meant to limit or otherwise define the scope of the invention.

In the detergent compositions, the abbreviated component identifications have the following meanings:

AExS: Sodium alkylether sulphate

LAS: Sodium linear C₁₂ alkyl benzene sulphonate

TAS: Sodium tallow alkyl sulphate

XYAS: Sodium C_{1X}-C_{1Y} alkyl sulfate

SS: Secondary soap surfactant of formula 2-butyl octanoic acid

25EY: A C₁₂-C₁₅ predominantly linear primary alcohol condensed with an average of Y moles of ethylene oxide

45EY: A C₁₄-C₁₅ predominantly linear primary alcohol condensed with an average of Y moles of ethylene oxide

XYEZS: C_{1X}-C_{1Y} sodium alkyl sulfate condensed with an average of Z moles of ethylene oxide per mole

Nonionic: C₁₃-C₁₅ mixed ethoxylated/propoxylated fatty alcohol with an average degree of ethoxylation of 3.8 and an average degree of propoxylation of 4.5 sold under the tradename Plurafax LF404 by BASF Gmbh

CFM: C₁₂-C₁₄ alkyl N-methyl glucamide

TFM: C₁₆-C₁₈ alkyl N-methyl glucamide

Silicate: Amorphous Sodium Silicate (SiO₂:Na₂O ratio=2.0)

NaSKS-6: Crystalline layered silicate of formula d-Na₂Si₂O₅

Carbonate: Anhydrous sodium carbonate

Phosphate: Sodium tripolyphosphate

MA/AA: Copolymer of 1:4 maleic/acrylic acid, average molecular weight about 80,000

Polyacrylate: Polyacrylate homopolymer with an average molecular weight of 8,000 sold under the tradename PA30 by BASF GmbH

Zeolite A: Hydrated Sodium Aluminosilicate of formula Na₁₂(AlO₂SiO₂)₁₂.27H₂O having a primary particle size in the range from 1 to 10 micrometers

Citrate: Tri-sodium citrate dihydrate

Citric: Citric Acid

Perborate: Anhydrous sodium perborate monohydrate bleach, empirical formula NaBO₂.H₂O₂

PB4: Anhydrous sodium perborate tetrahydrate

Percarbonate: Anhydrous sodium percarbonate bleach of empirical formula 2Na₂CO₃.3H₂O₂

TAED: Tetraacetyl ethylene diamine

CMC: Sodium carboxymethyl cellulose

DETPMP: Diethylene triamine penta (methylene phosphonic acid), marketed by Monsanto under the Tradename Dequest 2060

PVP: Polyvinylpyrrolidone polymer

EDDS: Ethylenediamine-N, N′-disuccinic acid, [S,S] isomer in the form of the sodium salt

Suds Suppressor: 25% paraffin wax Mpt 50° C., 17% hydrophobic silica, 58% paraffin oil

Granular Suds suppressor: 12% Silicone/silica, 18% stearyl alcohol, 70% starch in granular form

Sulphate: Anhydrous sodium sulphate

HMWPEO: High molecular weight polyethylene oxide

TAE 25: Tallow alcohol ethoxylate (25)

### DETERGENT EXAMPLE I

A granular fabric cleaning composition in accordance with the invention may be prepared as follows:

| | |
|---|---|
| Sodium linear C₁₂ alkyl | 6.5 |
| benzene sulfonate | |
| Sodium sulfate | 15.0 |
| Zeolite A | 26.0 |
| Sodium nitrilotriacetate | 5.0 |
| Enzyme of the invention | 0.1 |
| PVP | 0.5 |
| TAED | 3.0 |
| Boric acid | 4.0 |
| Perborate | 18.0 |
| Phenol sulphonate | 0.1 |
| Minors | Up to 100 |

### DETERGENT EXAMPLE II

A compact granular fabric cleaning composition (density 800 g/l) in accord with the invention may be prepared as follows:

| | |
|---|---|
| 45AS | 8.0 |
| 25E3S | 2.0 |
| 25E5 | 3.0 |
| 25E3 | 3.0 |
| TFAA | 2.5 |
| Zeolite A | 17.0 |
| NaSKS-6 | 12.0 |
| Citric acid | 3.0 |
| Carbonate | 7.0 |
| MA/AA | 5.0 |
| CMC | 0.4 |
| Enzyme of the invention | 0.1 |
| TAED | 6.0 |
| Percarbonate | 22.0 |
| EDDS | 0.3 |
| Granular suds suppressor | 3.5 |
| water/minors | Up to 100% |

### DETERGENT EXAMPLE III

Granular fabric cleaning compositions in accordance with the invention which are especially useful in the laundering of colored fabrics were prepared as follows:

| | | |
|---|---|---|
| LAS | 10.7 | — |
| TAS | 2.4 | — |
| TFAA | — | 4.0 |
| 45AS | 3.1 | 10.0 |
| 45E7 | 4.0 | — |
| 25E3S | — | 3.0 |
| 68E11 | 1.8 | — |
| 25E5 | — | 8.0 |
| Citrate | 15.0 | 7.0 |
| Carbonate | — | 10 |
| Citric acid | 2.5 | 3.0 |
| Zeolite A | 32.1 | 25.0 |
| Na-SKS-6 | — | 9.0 |
| MA/AA | 5.0 | 5.0 |
| DETPMP | 0.2 | 0.8 |
| Enzyme of the invention | 0.10 | 0.05 |
| Silicate | 2.5 | — |
| Sulphate | 5.2 | 3.0 |
| PVP | 0.5 | — |
| Poly (4-vinylpyridine)-N- | — | 0.2 |
| Oxide/copolymer of vinyl- | | |
| imidazole and vinyl- | | |
| pyrrolidone | | |
| Perborate | 1.0 | — |
| Phenol sulfonate | 0.2 | — |

| | |
|---|---|
| Water/Minors | Up to 100% |

### DETERGENT EXAMPLE IV

Granular fabric cleaning compositions in accordance with the invention which provide “Softening through the wash” capability may be prepared as follows:

| | | |
|---|---|---|
| 45AS | — | 10.0 |
| LAS | 7.6 | — |
| 68AS | 1.3 | — |
| 45E7 | 4.0 | — |
| 25E3 | — | 5.0 |
| Coco-alkyl-dimethyl hydroxy- | 1.4 | 1.0 |
| ethyl ammonium chloride | | |
| Citrate | 5.0 | 3.0 |
| Na-SKS-6 | — | 11.0 |
| Zeolite A | 15.0 | 15.0 |
| MA/AA | 4.0 | 4.0 |
| DETPMP | 0.4 | 0.4 |
| Perborate | 15.0 | — |
| Percarbonate | — | 15.0 |
| TAED | 5.0 | 5.0 |
| Smectite clay | 10.0 | 10.0 |
| HMWPEO | — | 0.1 |
| Enzyme of the invention | 0.10 | 0.05 |
| Silicate | 3.0 | 5.0 |
| Carbonate | 10.0 | 10.0 |
| Granular suds suppressor | 1.0 | 4.0 |
| CMC | 0.2 | 0.1 |

| | |
|---|---|
| Water/Minors | Up to 100% |

### DETERGENT EXAMPLE V

Heavy duty liquid fabric cleaning compositions in accordance with the invention may be prepared as follows:

| | I | II |
|---|---|---|
| LAS acid form | — | 25.0 |
| Citric acid | 5.0 | 2.0 |
| 25AS acid form | 8.0 | — |
| 25AE2S acid form | 3.0 | — |
| 25AE7 | 8.0 | — |
| CFAA | 5 | — |
| DETPMP | 1.0 | 1.0 |
| Fatty acid | 8 | — |
| Oleic acid | — | 1.0 |
| Ethanol | 4.0 | 6.0 |
| Propanediol | 2.0 | 6.0 |
| Enzyme of the invention | 0.10 | 0.05 |
| Coco-alkyl dimethyl | — | 3.0 |
| hydroxy ethyl ammonium | | |
| chloride | | |
| Smectite clay | — | 5.0 |
| PVP | 2.0 | — |

| | |
|---|---|
| Water/Minors | Up to 100% |

Uses in the Textile Industry

The pectate lyase enzyme of the present invention is useful in the cellulosic fiber processing industry for the pretreatment or retting of fibers from hemp, flax or linen.

The processing of cellulosic material for the textile industry, as for example cotton fiber, into a material ready for garment manufacture involves several steps: spinning of the fiber into a yarn; construction of woven or knit fabric from the yarn and subsequent preparation, dyeing and finishing operations. Woven goods are constructed by weaving a filling yarn between a series of warp yarns; the yarns could be two different types. Knitted goods are constructed by forming a network of interlocking loops from one continuous length of yarn. The cellulosic fibers can also be used for non-woven fabric.

The preparation process prepares the textile for the proper response in dyeing operations. The sub-steps involved in preparation are

a. Desizing (for woven goods) using polymeric size like e.g. starch, CMC or PVA is added before weaving in order to increase the warp speed; This material must be removed before further processing.

b. Scouring, the aim of which is to remove non-cellulosic material from the cotton fiber, especially the cuticle (mainly consisting of waxes) and primary cell wall (mainly consisting of pectin, protein and xyloglucan). A proper wax removal is necessary for obtaining a high wettability, being a measure for obtaining a good dyeing. Removal of the primary cell wall—especially the pectins—improves wax removal and ensures a more even dyeing. Further this improves the whiteness in the bleaching process. The main chemical used in scouring is sodium hydroxide in high concentrations, up to 70 g/kg cotton and at high temperatures, 80-95° C.; and

c. Bleaching; normally the scouring is followed by a bleach using hydrogen peroxide as the oxidizing agent in order to obtain either a fully bleached (white) fabric or to ensure a clean shade of the dye.

A one step combined scour/bleach process is also used by the industry. Although preparation processes are most commonly employed in the fabric state; scouring, bleaching and dyeing operations can also be done at the fiber or yarn stage.

The processing regime can be either batch or continuous with the fabric being contacted by the liquid processing stream in open width or rope form. Continuous operations generally use a saturator whereby an approximate equal weight of chemical bath per weight of fabric is applied to the fabric, followed by a heated dwell chamber where the chemical reaction takes place. A washing section then prepares the fabric for the next processing step. Batch processing generally takes place in one processing bath whereby the fabric is contacted with approximately 8-15 times its weight in chemical bath. After a reaction period, the chemicals are drained, fabric rinsed and the next chemical is applied. Discontinuous pad-batch processing involves a saturator whereby an approximate equal weight of chemical bath per weight of fabric is applied to the fabric, followed by a dwell period which in the case of cold pad-batch might be one or more days.

Woven goods are the prevalent form of textile fabric construction. The weaving process demands a “sizing” of the warp yarn to protect it from abrasion. Starch, polyvinyl alcohol (PVA), carboxymethyl cellulose, waxes and acrylic binders are examples of typical sizing chemicals used because of availability and cost. The size must be removed after the weaving process as the first step in preparing the woven goods. The sized fabric in either rope or open width form is brought in contact with the processing liquid containing the desizing agents. The desizing agent employed depends upon the type of size to be removed. For PVA sizes, hot water or oxidative processes are often used. The most common sizing agent for cotton fabric is based upon starch. Therefore most often, woven cotton fabrics are desized by a combination of hot water, the enzyme α-amylase to hydrolyze the starch and a wetting agent or surfactant. The cellulosic material is allowed to stand with the desizing chemicals for a “holding period” sufficiently long to accomplish the desizing. The holding period is dependent upon the type of processing regime and the temperature and can vary from 15 minutes to 2 hours, or in some cases, several days. Typically, the desizing chemicals are applied in a saturator bath which generally ranges from about 15° C. to about 55° C. The fabric is then held in equipment such as a “J-box” which provides sufficient heat, usually between about 55° C. and about 100° C., to enhance the activity of the desizing agents. The chemicals, including the removed sizing agents, are washed away from the fabric after the termination of the holding period.

In order to ensure a high whiteness or a good wettability and resulting dyeability, the size chemicals and other applied chemicals must be thoroughly removed. It is generally believed that an efficient desizing is of crucial importance to the following preparation processes: scouring and bleaching.

The scouring process removes much of the non-cellulosic compounds naturally found in cotton. In addition to the natural non-cellulosic impurities, scouring can remove dirt, soils and residual manufacturing introduced materials such as spinning, coning or slashing lubricants. The scouring process employs sodium hydroxide or related causticizing agents such as sodium carbonate, potassium hydroxide or mixtures thereof. Generally an alkali stable surfactant is added to the process to enhance solubilization of hydrophobic compounds and/or prevent their redeposition back on the fabric. The treatment is generally at a high temperature, 80° C.-100° C., employing strongly alkaline solutions, pH 13-14, of the scouring agent. Due to the non-specific nature of chemical processes not only are the impurities but the cellulose itself is attacked, leading to damages in strength or other desirable fabric properties. The softness of the cellulosic fabric is a function of residual natural cotton waxes. The non-specific nature of the high temperature strongly alkaline scouring process cannot discriminate between the desirable natural cotton lubricants and the manufacturing introduced lubricants. Furthermore, the conventional scouring process can cause environmental problems due to the highly alkaline effluent from these processes. The scouring stage prepares the fabric for the optimal response in bleaching. An inadequately scoured fabric will need a higher level of bleach chemical in the subsequent bleaching stages.

The bleaching step decolorizes the natural cotton pigments and removes any residual natural woody cotton trash components not completely removed during ginning, carding or scouring. The main process in use today is an alkaline hydrogen peroxide bleach. In many cases, especially when a very high whiteness is not needed, bleaching can be combined with scouring.

The following non-limiting examples illustrate the invention.

Materials and Methods

Strains and Donor Organism

Thermotoga maritima, DSM 3109.

B. subtilis DN1885 (Diderichsen, B., Wedsted, U., Hedegaard, L., Jensen, B. R., Sjøholm, C. (1990) Cloning of aldB, which encodes alpha-acetolactate decarboxylase, an exoenzyme from Bacillus brevis. J. Bacteriol., 172, 4315-4321). Competent cells were prepared and transformed as described by Yasbin, R. E., Wilson, G. A. and Young, F. E. (1975) Transformation and transfection in lysogenic strains of Bacillus subtilis: evidence for selective induction of prophage in competent cells. J. Bacteriol, 121:296-304.

E. coli DH10B (Life Technologies Ltd, England).

B. subtilis MB1053-1. This strain is PL 2306 in which the pectate lyase gene Pel has been disrupted resulting in a pectate lyase negative strain. The disruption was performed essentially as described in (Eds. A. L. Sonenshein, J. A. Hoch and Richard Losick (1993) Bacillus subtilis and other Gram-Positive Bacteria, American Society for microbiology, p. 618).

Competent cells were prepared and transformed as described by Yasbin, R. E., Wilson, G. A. and Young, F. E. (1975) Transformation and transfection in lysogenic strains of Bacillus subtilis: evidence for selective induction of prophage in competent cells. J. Bacteriol, 121:296-304.

B. subtilis PP289-5. This strain is described in U.S. Pat. No. 5,843,720, example 1, step 2C, issued on Dec. 1, 1998.

B. licheniformis SJ3047. This strain is described in PCT Patent application WO 99/41358. Essentially this strain is a amylase negative recombinant B. licheniformis strain.

Plasmids

pZErO-2 (Invitrogen, Calif., USA)

pMOL944:

This plasmid is a pUB110 derivative essentially containing elements making the plasmid propagatable in Bacillus subtilis, kanamycin resistance gene and having a strong promoter and signal peptide cloned from the amyL gene of B. licheniformis ATCC14580. The signal peptide contains a Sacil site making it convenient to clone the DNA encoding the mature part of a protein in-fusion with the signal peptide. This results in the expression of a Pre-protein directed towards the exterior of the cell.

The plasmid was constructed by means of conventional genetic engineering techniques briefly described in the following.

Construction of pMOL944:

The pUB 10 plasmid (McKenzie, T. et al., 1986, Plasmid 15:93-103) was digested with the unique restriction enzyme NciI. A PCR fragment amplified from the amyL promoter encoded on the plasmid pDN1981 (P. L. Jørgensen et al., 1990, Gene, 96, p 37-41.) was digested with NciI and inserted in the NciI digested pUB110 to give the plasmid pSJ2624.

The two PCR primers used have the following sequences:

| | |
|---|---|
| #LWN5494 | |
| 5′-GTCGCCGGGGCGGCCGCTATCAATTGGTAACTG | (SEQ ID NO:10) |
| TATCTCAGC-3′ | |
| #LWN5495 | |
| 5′-GTCGCCCGGGAGCTCTGATCAGGTACCAAGCTT | (SEQ ID NO:11) |
| GTCGACCTGCAGAATGAGGCAGCAAGAAGAT-3′ | |

The primer #LWN5494 inserts a NotI site in the plasmid.

The plasmid pSJ2624 was then digested with SacI and NotI and a new PCR fragment amplified on amyL promoter encoded on the pDN1981 was digested with SacI and NotI and this DNA fragment was inserted in the SacI-NotI digested pSJ2624 to give the plasmid pSJ2670.

This cloning replaces the first amyL promoter cloning with the same promoter but in the opposite direction. The two primers used for PCR amplification have the following sequences:

| | |
|---|---|
| #LWN5938 | |
| 5′-GTCGGCGGCCGCTGATCACGTACCAAGCTTGTC | (SEQ ID NO:12) |
| GACCTGCAGAATGAGGCAGCAAGAAGAT-3′ | |
| #LWN5939 | |
| 5′-GTCGGAGCTCTATCAATTGGTAACTGTATCTCA | (SEQ ID NO:13) |
| GC-3′ | |

The plasmid pSJ2670 was digested with the restriction enzymes PstI and BclI and a PCR fragment amplified from a cloned DNA sequence encoding the alkaline amylase SP722 (disclosed in the International Patent Application published as WO 95/26397 which is hereby incorporated by reference in its entirety) was digested with PstI and BclI and inserted to give the plasmid pMOL944. The two primers used for PCR amplification have the following sequence:

| | |
|---|---|
| #LWN7864 | |
| 5′-AACAGCTGATCACGACTGATCTTTTAGCTTGGC | (SEQ ID NO:14) |
| AC-3′ | |
| #LWN7901 | |
| 5′-AACTGCAGCCGCGGCACATCATAATGGGACAAA | (SEQ ID NO:15) |
| TGGG-3′ | |

The primer #LWN7901 inserts a SacI site in the plasmid.

General Molecular Biology Methods

Unless otherwise mentioned the DNA manipulations and transformations were performed using standard methods of molecular biology (Sambrook et al. (1989) Molecular cloning: A laboratory manual, Cold Spring Harbor lab., Cold Spring Harbor, N.Y.; Ausubel, F. M. et al. (eds.) “Current protocols in Molecular Biology”. John Wiley and Sons, 1995; Harwood, C. R., and Cutting, S. M. (eds.) “Molecular Biological Methods for Bacillus”. John Wiley and Sons, 1990).

Enzymes for DNA manipulations were used according to the specifications of the suppliers (e.g. restriction endonucleases, ligases etc. are obtainable from New England Biolabs, Inc.).

Media

TY (as described in Ausubel, F. M. et al. (eds.) “Current protocols in Molecular Biology”. John Wiley and Sons, 1995).

LB agar (as described in Ausubel, F. M. et al. (eds.) “Current protocols in Molecular Biology”. John Wiley and Sons, 1995).

LBPG is LB agar supplemented with 0.5% Glucose and 0.05 M potassium phosphate, pH 7.0

BPX media is described in EP 0 506 780 (WO 91/09129).

The End Point Lyase Assay (at 235 nm), Pectate Units.

For determination of the β-elimination an assay measuring the increase in absorbance at 235 nm was carried out using the substrate 1.0% polygalacturonic acid sodium salt (Sigma P-1879) solubilised in 0.1 M EPPS buffer pH 8. Incubation for 20 minutes at 70° C. The reaction is stopped by adding 5 volumes of 0.02 M H₃PO₄. For calculation of the catalytic rate an increase of 5.2 Absorbency at 235 units per min corresponds to formation of 1 μmol of unsaturated product (Nasuna and Starr (1966) J. Biol. Chem. Vol 241 page 5298-5306; and Bartling, Wegener and Olsen (1995) Microbiology Vol 141 page 873-881).

One Pectate Unit is the amount of enzyme resulting in formation of one micromole cleaved per minute at pH 8.0 and 70° C.

Genomic DNA Preparation

Strain Thermotoga maritima, DSM 3109, was propagated in Medium 343 at 80° C., anaerobically as specified by DSMZ (Deutsche Sammlung von Mikroorganismen und Zellkulturen GmbH (German Collection of Microorganisms and Cell Cultures)). After propagation the cells were harvested, and genomic DNA isolated by the method described by Pitcher et al. (Pitcher, D. G., Saunders, N. A., Owen, R. J. (1989). Rapid extraction of bacterial genomic DNA with guanidium thiocyanate. Lett. Appl. Microbiol., 8,151-156).

The following examples illustrate the invention.

### EXAMPLE 1

Cloning, Expression, Purification and Characterization of Pectate Lyase from Thermotoga maritima, DSM 3109

The pectate lyase encoded on the genome of Thermotoga maritima, (DSM 3109) (vide supra, represented by amino acid sequence SEQ ID NO:1) encoding DNA sequence of the invention was cloned as described below.

Genomic DNA of Thermotoga maritima, (DSM 3109) was used as template for PCR amplification using the primers #171130 and #171131 yielding a fragment of 1.0 kbp. The fragment was isolated on a 0.7% agarose gel and digested by the restriction enzymes SacI and NotI.

Primers:

| | |
|---|---|
| #171130: | |
| AAA CCG CGG CAT CTC TCA ATG ACA AAC | (SEQ ID NO:16) |
| CTG TGG G (SacII) | |
| #171131: | |
| AAA GCG GCC GCT GAG CCG TAT TTA GTT | (SEQ ID NO:17) |
| CTT CAA ACA CC (NotI) | |

The isolated DNA fragment was ligated to the Sacil and NotI digested plasmid pMOL944 (4.8 kbp), and the ligation mixture was used for transform Bacillus subtilis DN1885 (Diderichsen, B., Wedsted, U., Hedegaard, L., Jensen, B. R., Sjøholm, C. (1990) Cloning of aldB, which encodes alpha-acetolactate decarboxylase, an exoenzyme from Bacillus brevis. J. Bacteriol., 172, 4315-4321). Transformed cells from were plated on LB-agar containing 10 mM Potassium phosphate buffer pH 7.0, 0.4% glucose, 10 μg/ml kanamycin. The plated cells were incubated 16 hours at 37° C.

Several clones were re-streaked on fresh agar plates and also grown in liquid TY cultures with 10 μg/ml kanamycin and incubated overnight at 37° C. Next day 1 ml of cells were used to isolate plasmid from the cells using the Qiaprep Spin Plasmid Miniprep Kit #27106 according to the manufacturers recommendations for B. subtilis plasmid preparations. One correct construct was saved as the clone JA855. This plasmid DNA (pJA855) was used as template for DNA sequencing. The DNA sequence thus revealed is represented by DNA sequence SEQ ID NO:2. This sequence is a fusion between the DNA encoding the signal peptide cloned from the amyL gene of B. licheniformis ATCC14580 and the DNA encoding the mature part of the pectate lyase of Thermotoga maritima, wherein positions 1 to 87 are the AmyL-signal; positions 88 to 1107 encode the Thermotoga maritima pectate lyase mature part, and positions 1108 to 1149 are vector pMOL944 DNA.

The derived protein sequence of SEQ ID NO:2 is represented in SEQ ID NO:3 where positions 1 to 29 are the AmyL signal peptide, positions 30 to 369 are the mature pectate lyase, and positions 370 to 383 the expressed vector.

The cloned DNA sequence was expressed in B. subtilis by fermenting the JA855 cells in BP-X media containing 10 μg/ml of kanamycin at 37° C. for 5 days at 300 rpm.

### EXAMPLE 2

Purification and Characterisation of Pectate Lyase Cloned from Thermotoga maritima (JA855)

The clone JA855 obtained as described in example 1 was incubated in 2200 ml of BPX containing mg/ml kanamycin from shake flasks with a final pH of 7.2.

The fermentation medium was diluted with one volume of water and flocculated using cationic flocculation agent C521 (10% solution) and 0.1% solution of anionic agent A130: To 4000 ml of broth was added 170 ml of C521 (10%) simultaneous with 380 ml of A130 under stirring at room temperature. The flocculated material was separated by centrifugation using a Sorval RC 3B centrifuge at 4,500 rpm for 30 minutes. The supernatant was clarified using Whatman glass filter number F. In total was obtained 4200 ml of clear solution containing 100,000 Pectate Units.

The liquid was concentrated into 400 ml using filtron ultrafiltration with a MW cut-off of 10 kDa.

For obtaining a pure enzyme 2 ml of this partial pure enzyme was applied to a size chromatography (Superdex 200) column equilibrated with 0.1 M sodium acetate, pH 6.0. The pectate lyase eluted as a single peak with a MW of 38 kDa in SDS-PAGE and with a specific activity of 50 Pectate Units per mg protein.

The cloned pectate lyase of the invention was used for raising rabbit antiserum.

After electroblotting of this band the N-terminal was determined as:

ASLNDKPVGFASVP

This is in agreement with the amino acid sequence shown in SEQ ID NO:3 deduced from the DNA sequence shown in SEQ ID NO:2 with a 28 amino acid prosequence. The calculated MW from the deduced sequence was 38 kDa and the calculated pI was 5. The molar extinction coefficient at 280 nm was 37,460.

The β-transelimination activity (using the lyase assay at 235 nm) at different pH values was determined as steady state kinetic at 70° C., substrate 1.0% polygalacturonic acid sodium salt (Sigma P-1879). The relative rate is calculated as percentage of the optimum activity, the following result was obtained:

| PH | % Activity |
|---|---|
| 4.5 | 1 |
| 5.0 | 1 |
| 5.4 | 4 |
| 6.9 | 7 |
| 7.4 | 11 |
| 7.8 | 9 |
| 8.3 | 19 |
| 8.5 | 11 |
| 9.3 | 34 |
| 9.9 | 81 |
| 10.2 | 100 |

Correspondingly, the relative activity at different temperatures (at pH 9; 0.39 millimoles CaCl₂; substrate 1.0% polygalacturonic acid sodium salt (Sigma P-1879)) was found:

| Temp. ° C. | % Activity |
|---|---|
| 70 | 38 |
| 80 | 54 |
| 90 | 76 |
| 100 | 100 |

Correspondingly, the relative activity at different temperatures (at pH 8; 0.39 mmol CaCl₂; substrate 1.0% polygalacturonic acid sodium salt (Sigma P-1879)) was found:

| Temp. ° C. | % Activity |
|---|---|
| 70 | 22 |
| 80 | 48 |
| 90 | 81 |
| 100 | 100 |

CaCl₂ dependency at pH 8.0 and 70° C.: Optimum activity was obtained using between 0.1 and 0.6 millimoles CaCl₂. Excess EDTA inhibited the catalytic activity.

DSC in sodium acetate buffer at pH 6.0 showed a melting temperature around 98° C.

### EXAMPLE 3

Cloning and Verification of Thermotoga maritima Pectate Lyase in E. coli

Cloning of the Thermotoga maritima Pectate Lyase

The pectate lyase Thermotoga maritima, DSM 3109, was cloned by PCR cloning applying the following primers: GGGAATTCTTACTGAGCCGTATTTAGTTC (SEQ ID NO:18) and CCGGATCCAGTAGGGAGGGATGCTCATG (SEQ ID NO:19) on purified chromosomal DNA. The purified PCR fragment was digested with EcoRI and BamHI, cloned into pZErO-2 (Invitrogen, Calif., USA) restricted with EcoRI and BamHI and transformed into E. coli DH10B (Life Technologies Ltd, England). Several clones were sequenced and one clone with the right sequence encoding the Thermotoga maritima pectate lyase was given the name PEC1038. This DNA sequence is represented as the SEQ ID NO:4 and the derived protein sequence is represented as the SEQ ID NO:5. Molecular biology was conducted using methods known to persons skilled in the art.

Functional Analysis:

The pectate lyase of Thermotoga maritima was functionally characterized as follows: The PEC1038 clone was inoculated into TY medium with 25 μg/ml kanamycin, grown to OD₄₉₀˜0.6 when isopropyl β-D-thiogalactopyranoside (Sigma) was added to the final concentration of 1 mM. The growth was continued at 37° C. overnight before samples were tested for pectinase activity.

Fluorescence polarisation (PCT/DK99/00112) was applied in order to monitor the pectinase activity, as this technology is compatible with the elevated temperature. Assay mixes consisting of 100 μl overnight cultures (PEC1038 was used as positive sample and DH10B as negative control) and 400 μl assay buffer (50 mM Hepes pH 8, 25 μg/ml fluorescein labelled pectin (DE 3%) (PCT/DK99/00112) and 1 mM CaCl₂) was incubated in Eppendorf tubes placed in heating blocks set at various temperatures (See Table 1). Mineral oil was applied on top of the assay mixes in order to avoid evaporation. The Bacillus agaradhaerens pectate lyase (cf. WO 99/27084, SEQ ID NO:2) was included as positive control and was applied at the final concentration of 5.5 μg/ml.

Values in Table 1 are given as relative changes in fluorescence polarisation value corrected for the background (DH10B sample). As can be seen, end-point reaction resulted in a decrease in the polarisation value of approximately 23%. The PEC1038 samples did only reach end-point reaction at elevated temperatures (80° C. and 90° C.) as supposed to 9% decrease at 60° C. after 3 hours incubation which demonstrates that the Thermotoga pectate lyase is relatively more active at 80° C. and 90° C. compared to 60° C. The experiment also demonstrates that the pectate lyase from Thermotoga maritima is more active at 90° C. than at 80° C. (See data for 1 and 2 hours incubation).

| | | | |
|---|---|---|---|
| 60° C. | 70° C. | 80° C. | 90° C. |

1 hour

| | | | | |
|---|---|---|---|---|
| PEC1038 | 4% | 1% | 8% | 16% |
| B. agradhaerens | 23% | | | |
| pectate lyase | | | | |

2 hours

| | | | | |
|---|---|---|---|---|
| PEC1038 | 8% | 6% | 9% | 20% |
| B. agaradhaerens | 23% | | | |
| pectate lyase | | | | |

3 hours

| | | | | |
|---|---|---|---|---|
| PEC1038 | 9% | 19% | 24% | 21% |
| B. agaradhaerens | 24% | | | |
| pectate lyase | | | | |

Table 1: Activity of PEC1038 and B. agaradhaerens pectate lyase, shown as relative decreases given in percentage of the polarisation value of the negative control DH10B). Samples were incubated at 60° C., 70° C., 80° C. and 90° C. for 1, 2 and 3 hours.

### EXAMPLE 4

Cloning of Native Pectate Lyase from Thermotoga maritima, DSM 3109

The pectate lyase encoded on the genome of Thermotoga maritima, DSM 3109 (vide supra, represented by amino acid sequence SEQ ID NO:1) encoding DNA sequence of the invention was cloned as described below.

Genomic DNA of Thermotoga maritima, DSM 3109, was used as template for PCR amplification. The oligonucleotides #185245 and #186757 were used in a PCR reaction in HiFidelity™ PCR buffer (Boehringer Mannheim, Germany) supplemented with 200 μM of each dNTP, 2.6 units of HiFidelity™ Expand enzyme mix and 200 pmol of each primer.

Primers:

| | |
|---|---|
| #185245: | |
| 5′-CAT TCT GCA GCC GCG GCA TCT CTC | (SEQ ID NO:20) |
| AAT GAC AAA CCT GTG GG-3′ (SacII) | |
| #186757: | |
| 5′-CAT CAT GGA TCC GCG GCC GCT TAT | (SEQ ID NO:21) |
| CAC TGA GCC GTA TTT AGT TCT TCA AAC- | |
| 3′ (NotI) | |

The PCR reaction was performed using a DNA thermal cycler (Landgraf, Germany). One incubation at 94° C. for 1 min followed by ten cycles of PCR performed using a cycle profile of denaturation at 94° C. for 15 sec, annealing at 60° C. for 60 sec, and extension at 72° C. for 120 sec, followed by twenty cycles of denaturation at 94° C. for 15 sec, 60° C. for 60 sec and 72° C. for 120 sec (at this elongation step 20 sec are added every cycle). Five μl aliquots of the amplification product was analysed by electrophoresis in 0.7% agarose gels (NuSieve, FMC). The appearance of a DNA fragment size 1.0 kb indicated proper amplification of the gene segment.

Subcloning of PCR Fragment:

Forty five μl aliquots of the PCR products generated as described above were purified using QIAquick PCR purification kit (Qiagen, USA) according to the manufacturer's instructions. The purified DNA was eluted in 50 μl of 10 mM Tris-HCl, pH 8.5.

5 μg of pMOL944 and twenty five μl of the purified PCR fragment was digested with SacI and NotI, electrophoresed in 0.7% agarose gels (NuSieve, FMC), the relevant fragments were excised from the gels, and purified using QIAquick Gel extraction Kit (Qiagen, USA) according to the manufacturer's instructions. The isolated PCR DNA fragment was then ligated to the SacI-NotI digested and purified pMOL944. The ligation was performed overnight at 16° C. using 0.5 μg of each DNA fragment, 1 U of T4 DNA ligase and T4 ligase buffer (Boehringer Mannheim, Germany).

The ligation mixture was used for transform Bacillus subtilis MB1053-1. Transformed cells from were plated on LB-agar containing 10 mM potassium phosphate buffer pH 7.0, 0.4% glucose, 10 μg/ml kanamycin. The plated cells were incubated for 16 hours at 37° C.

Several clones were re-streaked on fresh agar plates and also grown in liquid TY cultures with 10 μg/ml kanamycin and incubated overnight at 37° C. Next day 1 ml of cells were used to isolate plasmid from the cells using the Qiaprep Spin Plasmid Miniprep Kit #27106 according to the manufacturers recommendations for B. subtilis plasmid preparations. One correct construct was saved as the clone MB1083. This plasmid DNA, pMB1083 was used as template for DNA sequencing. The DNA sequence thus revealed is represented by DNA sequence SEQ ID NO:6. This sequence is a fusion between the DNA encoding the signal peptide cloned from the amyL gene of B. licheniformis ATCC14580 and the DNA encoding the mature part of the pectatelyase of Thermotoga maritima, wherein positions 1 to 84 represent the AmyL-signal and positions 85 to 1107 are the Thermotoga maritima pectate lyase mature part.

The derived protein sequence of SEQ ID NO:6 is represented in SEQ ID NO:7 where positions 1 to 29 are the AmyL signal peptide and positions 30 to 369 are the mature pectate lyase.

The cloned DNA sequence was expressed in B. subtilis by fermenting the MB1083 cells in BP-X media containing 10 μg/ml of kanamycin, at 37° C. for 5 days at 300 rpm.

### EXAMPLE 5

Expression of Thermotoga maritima pectate lyase in B. licheniformis

Plasmid pMB1083 was used to transform competent cells of B. subtilis PP289-5 (dal-, pLS20, pBC16; U.S. Pat. No. 5,843,720, example 1, step 2C) selecting kanamycin (10 μg/ml) and tetracycline (5 pg/ml) resistance at 30° C. on LBPG agar plates supplemented with D-alanine (100 μg/ml). One transformant was kept, MB1101.

The donor strain MB1101 was used to transfer its plasmid into B. licheniformis by is conjugation, essentially as described in U.S. Pat. No. 5,843,720, example 1, step 2D. Transconjugants were selected on LBPG 10 μg/ml Kanamycin plates. One transconjugant was kept, MB1105.

MB1105 was grown overnight in LB media, plasmid DNA was isolated and characterized, the characterization revealed that the original plasmid, pMB1083, had been established in B. licheniformis. The recombinant Thermotoga maritima pectate lyase expressed from MB1105 was expressed, purified and characterized as described below.

### EXAMPLE 6

Purification and Characterisation of Pectate Lyase Cloned from Thermotoga maritima and Expressed in Bacillus licheniformis (clone MB1105)

The clone MB1105 obtained as described in example 6 was incubated in shake flasks using the substrate PS1 with 10 μg/l kanamycin and total 3100 ml fermentation broth was obtained from the shake flasks.

The fermentation medium was adjusted to pH 7.5 and 31 ml of 50% CaCl₂ was added. Then 31 ml of 11% freshly made solution of sodium aluminates was added using a pH titration and 20% formic acid for keeping the pH at 7.5. Finally the cells were flocculated using cationic flocculation agent C521 (10% solution) and 0.1% solution of anionic agent A130: 78 ml of C521 (10%) was added simultaneously with 233 ml of A130 under stirring at room temperature. The flocculated material was separated by centrifugation using a Sorval RC 3B centrifuge at 4,500 rpm for 20 minutes. The supernatant was clarified using Whatman glass filter number F. In total was obtained 2500 ml of clear solution containing 280,000 Pectate Units.

The liquid was concentrated into 360 ml, using filtron ultra filtration with a MW cut off of 10 kDa. The solution was diluted to 1450 ml using ionized water and applied to a Q-Sepharose column equilibrated with 25 mM Tris pH 7.5. The pectate lyase was eluted from the column using a sodium chloride gradient. For obtaining a pure enzyme 2 ml of this partial pure enzyme was applied to a size chromatography (Superdex 200) column equilibrated with 0.1 M sodium acetate pH 6.0. The pectate lyase eluted as a single peak with a MW of 38 kDa in SDS-PAGE.

### EXAMPLE 7

Cloning, Expression of a Variant of Pectate Lyase from Thermotoga maritima, DSM 3109

The DNA sequence of the Thermotoga maritima pectate lyase was altered in such a way that three cysteine codons were changed into three other amino acids, the resulting DNA sequence and derived protein sequence are found in SEQ ID NO:8 and SEQ ID NO:9, respectively.

Plasmid DNA pJA855 was used as template for PCR amplification. The oligonucleotides #185245 and #186339 were used in a PCR reaction in HiFidelity™ PCR buffer (Boehringer Mannheim, Germany) supplemented with 200 μM of each dNTP, 2.6 units of HiFidelity™ Expand enzyme mix and 200 pmol of each primer. In another PCR reaction the oligonucleotides #186757 and #186340 were used in a PCR reaction in HiFidelity™ PCR buffer (Boehringer Mannheim, Germany) supplemented with 200 pM of each dNTP, 2.6 units of HiFidelity™ Expand enzyme mix and 200 pmol of each primer. The two resulting PCR fragments were purified using QIAquick PCR purification kit (Qiagen, USA) according to the manufacturer's instructions. The purified DNA was eluted in 50 μl of 10 mM Tris-HCl, pH 8.5.

These two PCR fragments were assembled in a third PCR reaction called a SOE PCR, equimolar amounts of the two PCR fragments were set up in a PCR reaction in HiFidelity™ PCR buffer (Boehringer Mannheim, Germany) supplemented with 200 μM of each dNTP, 2.6 units of HiFidelity™ Expand enzyme mix and 200 pmol of each of the primers #185245 and #186757.

Primers:

| | |
|---|---|
| #185245: | |
| 5′-CAT TCT GCA GCC GCG GCA TCT CTC | (SEQ ID NO:22) |
| AAT GAC AAA CCT GTG GG-3′ (SacII) | |
| #186339: | |
| 5′-CCA ACG AGG GAG ACC TTG TCG TGA | (SEQ ID NO:23) |
| TCC ACA AAT TTG YTC CAG GAA ACA GTG | |
| ATG TAG TTT GAG TAT TTT TTA ATA TCC | |
| ACT GCA CCA TCG TTG CCG TTG ACG AAG | |
| GTA WYG TGG TCG-3′ | |
| #186340: | |
| 5′-GGA TCA CGA CAA GGT CTC CCT CGT | (SEQ ID NO:24) |
| TGG TTC CTC CGA CAA AGA AGA TCC GGA | |
| ACA GGC AGG GCA GGC TTA CAA GGT CAC | |
| GTA CCA CCA TAA CTA CTT CAA GAA CCT | |
| GAT TCA GAG-3′ | |
| #186757: | |
| 5′-CAT CAT GGA TCC GCG GCC GCT TAT | (SEQ ID NO:25) |
| CAC TGA GCC GTA TTT AGT TCT TCA AAC- | |
| 3′ (NotI) | |

The PCR reactions were performed using a DNA thermal cycler (Landgraf, Germany). One incubation at 94° C. for 1 min followed by ten cycles of PCR performed using a cycle profile of denaturation at 94° C. for 15 sec, annealing at 60° C. for 60 sec, and extension at 72° C. for 120 sec, followed by twenty cycles of denaturation at 94° C. for 15 sec, 60° C. for 60 sec and 72° C. for 120 sec (at this elongation step 20 sec are added every cycle). Five μl aliquots of the amplification product was analysed by electrophoresis in 0.7% agarose gels (NuSieve, FMC).

Subcloning of PCR Fragment:

Forty five μl aliquots of the third PCR product (from the SOE PCR) generated as described above were purified using QIAquick PCR purification kit (Qiagen, USA) according to the manufacturer's instructions. The purified DNA was eluted in 50 μl of 10 mM Tris-HCl, pH 8.5.

5 μg of pMOL944 and twenty five μl of the purified PCR fragment was digested with SacI and NotI, electrophoresed in 0.7% agarose gels (NuSieve, FMC), the relevant fragments were excised from the gels, and purified using QIAquick Gel extraction Kit (Qiagen, USA) according to the manufacturer's instructions. The isolated PCR DNA fragment was then ligated to the SacI-NotI digested and purified pMOL944. The ligation was performed overnight at 16° C. using 0.5 μg of each DNA fragment, 1 U of T4 DNA ligase and T4 ligase buffer (Boehringer Mannheim, Germany).

The ligation mixture was used for transforming Bacillus subtilis MB1053-1. Transformed cells were plated on LB-agar containing 10 mM potassium phosphate buffer pH 7.0, 0.4% glucose, 10 μg/ml kanamycin. The plated cells were incubated for 16 hours at 37° C.

Several clones were re-streaked on fresh agar plates and also grown in liquid TY cultures with 10 μg/ml kanamycin and incubated overnight at 37° C. Next day 1 ml of cells were used to isolate plasmid from the cells using the Qiaprep Spin Plasmid Miniprep Kit #27106 according to the manufacturers recommendations for B. subtilis plasmid preparations. One construct was saved as the clone MB1081. This plasmid DNA, pMB1081, was used as template for DNA sequencing. The DNA sequence thus revealed is represented by DNA sequence SEQ ID NO:8. This sequence is a fusion between the DNA encoding the signal peptide cloned from the amyL gene of B. licheniformis ATCC14580 and the DNA encoding the mature part of the pectate lyase of Thermotoga maritima, wherein three of the cysteine codons are altered to three other amino acid codons. In the sequence, positions 1 to 84 are the AmyL-signal, and positions 85 to 1107 are the Thermotoga maritima pectate lyase mature part with three cysteine codons altered.

The derived protein sequence of SEQ ID NO:8 is represented in SEQ ID NO:9 wherein positions 1-29 are AmyL signal peptide; postions 30-369 are the mature pectate lyase with three cysteins altered.

The cloned DNA sequence was expressed in B. subtilis by fermenting the MB1081 cells in BP-X media containing 10 μg/ml of kanamycin, at 37° C. for 5 days at 300 rpm.

### EXAMPLE 8

Expression of Thermotoga maritima Pectate Lyase in B. licheniformis

Plasmid pMB1081 was used to transform competent cells of B. subtilis PP289-5 (dal-, pLS20, pBC16; U.S. Pat. No. 5,843,720, example 1, step 2C) selecting kanamycin (10 μg/ml) and tetracycline (5 μg/ml) resistance at 30° C. on LBPG agar plates supplemented with D-alanine (100 μg/ml). One transformant was kept, MB1000.

The donor strain MB1000 was used to transfer its plasmid into B. licheniformis by conjugation, essentially as described in U.S. Pat. No. 5,843,720, example 1, step 2D. Transconjugants were selected on LBPG 10 μg/ml Kanamycin plates. One transconjugant was kept, MB1104.

MB1104 was grown overnight in LB media, plasmid DNA was isolated and characterized, the characterization revealed that the original plasmid, pMB1081, had been established in B. licheniformis. The recombinant Thermotoga maritima pectate lyase expressed from MB1104 was expressed, purified and characterised as described below.

### EXAMPLE 9

Purification and Characterisation of Pectate Lyase Cloned from Thermotoga maritima, Site Directed Variant with 3 Cysteines Substituted, and Expressed in Bacillus licheniformis (Clone MB1104)

The clone MB1104 obtained as described in example 8 was incubated in 500 ml shake flasks containing 100 ml BP-X media with 10 μg/l kanamycin a total of 3300 ml fermentation broth was obtained from shake flasks.

The fermentation medium was adjusted to pH 7.5 and 33 ml 50% CaCl₂ was added. Then 33 ml of 11% freshly made solution of sodium aluminates was added using a pH titration and 20% formic acid for keeping the pH at 7.5. Finally the cells were flocculated using cationic flocculation agent C521 (10% solution) and 0.1% solution of anionic agent A130: 83 ml of C521 (10%) was added simultaneously with 248 ml of A130 under stirring at room temperature. The flocculated material was separated by centrifugation using a Sorval RC 3B centrifuge at 4,500 rpm for 20 minutes. The supernatant was filtrated using Whatman glass filters GF/D and F. The clear sterile solution was concentrated on a Filtron with a MW cut-off at 10 kDa, and the concentrate was diluted with ion-free water for finally to obtain a solution with a conductivity of 3 mSi in a volume of 1000 ml and the pH adjusted to 7.5. This was then applied to Q-Sepharose column equilibrated with 25 mM Tris pH 7.5. The pectate lyase bound to the ion-exchange column and was eluted using a NaCl gradient. The pure enzyme was 90% pure in SDS-PAGE with a main band at 38 kDa. The enzyme was further purified using Sephadex.

The purified pectate lyase from MB1104 was analyzed for its activity in different buffers representing different pH. The buffers used were: Na-MES 0.1 M pH 6.0; Na-MOPS 0.1 M pH 6.5, Na-MOPS 0.1 M pH 7.0; Phosphate 0.1 M pH 7.5; EPPS 0.1 M pH 8.0; EPPS 0.1 M pH 8.5; Na.glycine 0.1 M pH 9.0; Na.glycine 0.1 M pH 9.5; Na.glycine 0.1 M pH 10.0 and Na.glycine 0.1 M pH 10.5. Pectate lyase activity was determined as described above in the section “The End Point Lyase assay (at 235 nm), Pectate Units.” The incubation temperature in this investigation was 70° C.

The β-transelimination activity (using the lyase assay at 235 nm) at different pH values was determined as steady state kinetic at 70° C., (at pH 8; 0.68 millimoles CaCl₂; substrate 1.0% polygalacturonic acid sodium salt (Sigma P-1879)). The relative rate is calculated as percentage of the optimum activity, the following result was obtained:

| pH | % Activity |
|---|---|
| 6.0 | 9.0 |
| 6.5 | 13.4 |
| 7.0 | 18.6 |
| 7.5 | 63.2 |
| 8.0 | 33.9 |
| 8.5 | 47.0 |
| 9.0 | 29.2 |
| 9.5 | 43.3 |
| 10 | 86.6 |
| 10.5 | 100 |

The purified pectate lyase from MB1104 was analyzed for its activity under different temperatures the bufer system was 0.1 M EPPS pH 8.0 and temperatures investigated was: 70° C., 80° C., 90° C. and 95° C. Pectate lyase activity was determined as described above in the section “The End Point Lyase assay (at 235 nm), Pectate Units.” In the table below the activity measured at 95° C. was set to a 100%.

Similar to the determination of the pH profile, the relative activity at different temperatures (at pH 8; 0.68 millimoles CaCl₂; substrate 1.0% polygalacturonic acid sodium salt (Sigma P-1879)) was found:

| Temp. ° C. | % Activity |
|---|---|
| 70 | 39 |
| 80 | 53 |
| 90 | 69 |
| 95 | 100 |

1. A bacterial host cell transformed with a vector comprising a DNA sequence that is endogenous to a strain of Thermotoga maritima or a variant of the DNA sequence, which DNA sequence or variant DNA sequence encodes for a pectate lyase polypeptide (EC 4.2.2.2).

2. The host cell of claim 1, wherein the strain of Thermotoga maritima is the strain Thermotoga maritima, DSM 3109.

3. The host cell of claim 1 which is neutralophilic, alkalophilic, mesophilic or thermophilic.

4. The host cell of claim 1 which is a Bacillus host cell.

5. The host cell of claim 4, which is a Bacillus amyloliquefaciens, Bacillus licheniformis, Bacillus megaterium, Bacillus stearothermophilus or Bacillus subtilisr cell.

6. The host cell of claim 1, wherein the vector is integrated into the genome of the host.

7. The host cell of claim 1, wherein the vector is integrated into the genome of the untransformed host.

8. The host cell of claim 1, wherein the vector is present as an expression plasmid.

9. The host cell of claim 8, wherein the vector has been amplified on the genome or the expression plasmid is a multi-copy plasmid.

10. A bacterial expression vector which carries an inserted DNA sequence encoding for a pectate lyase polypeptide (EC 4.2.2.2) endogenous to a strain of Thermotoga maritima or a variant of the pectate lyase polypeptide.

11. The vector of claim 10 in which the expression cassette comprises regulatory regions from a species of Bacillus.

12. The vector of claim 11, wherein the Bacillus sp. regulatory regions are endogeneous to the host.

13. A method for producing a pectate lyase (EC 4.2.2.2) polypeptide endogenous to a strain of Thermotoga maritima or a variant of the pectate lyase polypeptide, the method comprising the steps of:
(a) growing a bacterial host cell in a nutrient medium, under conditions to overproduce the pectate lyase polypeptide, wherein the bacterial host cell has been en transformed with an expression cassette which includes, as operably joined components,
(i) a transcriptional and translational initiation regulatory region,
(ii) a DNA sequence encoding the pectate lyase polypeptide,
(iii) a transcriptional and translational termination regulatory region, wherein the regulatory regions are functional in the host, and
(iv) a selection marker gene for selecting transformed host cells; and
(b) recovering the pectate lyase polypeptide.

14. A polypeptide having pectate lyase activity (EC 4.2.2.2), which polypeptide is selected from the group consisting of
(a) polypeptides having pectate lyases activity, wherein the polypeptide is encoded by a DNA sequence endogenous to a strain of Thermotoga maritima; and
(b) site directed variants of the polypeptide encoded by a DNA sequence endogenous to a strain of Thermotoga maritima, wherein one, two, three or four cysteine residues have been altered to other amino acid residues.

15. The polypeptide of claim 14, wherein three cysteine residues have been altered to other amino acid residues.

16. The polypeptide of claim 15, wherein the cysteine residues independently of each other have been altered to asparagines, isoleucine or leucine.

17. The polypeptide of claim 14, wherein the strain of Thermotoga maritima is the strain Thermotoga maritima, DSM 3109.

18. The polypeptide of claim 16, which variant has amino acid substitutions in positions 161, 185 and 223 relative to the amino acid numbering of SEQ ID NO: 3.

19. The polypeptide of claim 16, which variant has a catalytically active domain represented by positions 30 to 369 of SEQ ID NO: 9.

20. A method for optimizing pectate lyase expression in a bacterial host, the method comprising the steps of:
(a) in the host, expressing a pectate lyase polypeptide fused to a reporter molecule;
(b) in the supernatant of the fermented host, monitoring the concentration of expressed pectate lyase polypeptide by measuring the intrinsic property or properties of the reporter molecule.

21. The method of claim 20, wherein the reporter molecule is a Green Fluorescent Protein, and the intrinsic property is fluorescence emission.

22. A polypeptide hybrid consisting essentially of a pectate lyase polypeptide fused to a green fluorescent protein.

23. A method of producing the hybrid of claim 22, comprising (a) growing a transformed host under conditions to express the hybrid whereby the transformed culture is substantially free of untransformed cells; (b) incubating the transformed culture in a nutrient medium, whereby the hybrid is overproduced; and (c) recovering the hybrid.

24. A detergent composition comprising the polypeptide of claim 14 and a surfactant.

25. A process for machine treatment of a fabric, comprising treating the fabric during a washing cycle of a machine washing process with a washing solution containing the polypeptide of claim 14 and a surfactant.

26. The process of claim 25, wherein the fabric is made of fibers selected from the group consisting of hemp, jute, flax and linen.

27. The process of claim 26, wherein the washing solution is added during a textile scouring process step.
